(19) United States
(12) Patent Application Publication
(10) Pub. No.: US 2023/0045044 A1
(43) Pub. Date: Feb. 9, 2023

(54) SURGICAL SIMULATION NETWORK SYSTEM

(71) Applicant: Applied Medical Resources Corporation, Rancho Santa Margarita, CA (US)

(72) Inventors: Mikhail Ovchinnikov, Rancho Santa Margarita, CA (US); Chris Meyer, Irvine, CA (US); Chris Thai, Irvine, CA (US); Hudson Albert, Rancho Santa Margarita, CA (US); Branden Carter, Rancho Santa Margarita, CA (US); Tong Choi, Irvine, CA (US); Kristen Lam, Los Angeles, CA (US); Aditya Kumakale, Rancho Santa Margarita, CA (US); Irfan Kil, Rancho Santa Margarita, CA (US)

(21) Appl. No.: 17/880,578

(22) Filed: Aug. 3, 2022

Related U.S. Application Data
(60) Provisional application No. 63/229,011, filed on Aug. 3, 2021.

(51) Int. Cl. A61B 90/00; G09B 23/28

(52) U.S. Cl. CPC A61B 90/36; G09B 23/285

(57) ABSTRACT

A surgical simulation network system is provided that facilitates user training in various surgical procedures and subsequent assessments of the user by allowing the users and assessors to be at different physical locations. The surgical simulation network system is part of a network having various surgical trainers that capture user performance data related to various surgical simulations. The user performance data is subsequently accessible by one or more assessors via the surgical simulation network system that allows the assessors to assess the user's competency. Thus, the users and the assessors can be located at different locations. Furthermore, the performance of the simulation and the assessments can be performed at different times.

### CROSS-REFERENCE TO RELATED APPLICATIONS

This application claims benefit of U.S. Provisional Patent Application No. 63/229,011, filed on Aug. 3, 2021, the disclosure of which are hereby incorporated by reference in their entirety.

### BACKGROUND

The present application generally relates to surgical training systems, and, more particularly, to simulated surgical training systems for teaching, practicing, and assessing various surgical techniques and procedures related but not limited to laparoscopic, endoscopic, and minimally invasive surgery.

Despite the continual rise in surgical care, many surgical procedures are rarely or lightly encountered during medical training. As such, there is an apparent growing concern amongst the surgical community regarding the sufficient availability and exposure to surgical training. Challenges thus remain for trainees who desire accurate, repeatable, and accessible training prior to entering the operation room. Likewise, there are challenges for assessors of such trainees to access such trainee surgical performances as well as accurately and repeatably apply such assessments and further instruction and training. Furthermore, there lacks a singular simulation system that overcomes these challenges.

### SUMMARY

In accordance with various embodiments of the present invention, a surgical simulation network is provided that has one or more surgical trainers that are configured to capture data pertaining to a user's performance of one or more surgical simulations at a respective surgical trainer. One or more of the surgical trainers are communicatively connected to a surgical simulation system where the data is streamed/transmitted to. The surgical simulation system is capable of processing and storing the user data so that it can be retrieved at some later time by one or more accessors. The accessors are able to access the user performance data in order to review and assess the user's performance. In various embodiments, the assessors can provide feedback related to the user's performance; the feedback which can be viewable by the user. The users and surgical trainers can be at different locations compared to where the assessors are located doing the review and assessment of the user performance. Furthermore, the assessors can view the user performance data at a different time than when the user performs the surgical simulation and still be capable of reviewing and providing feedback accordingly thereby providing flexibility on who and when feedback can be provided.

In various embodiments, the user performance data can be processes using computer vision and/or machine learning (A.I.) to facilitate the assessment. For example, computer vision and/or machine learning (A.I.) is capable of providing user assessments alongside the feedback provided from assessors. In addition, different guidelines can be provided for assessing a user's performance based on the experience (i.e. expert or novice) of the user.

In accordance with various embodiments of the present invention, a surgical simulation network is provided that has a memory module that includes a program that is executable by a processor that performs a method that facilitates user assessments. The method includes steps of receiving user performance data from one or more surgical trainers that are communicatively connected to the surgical simulation network, the user performance data corresponding to a user performance of a surgical simulation, providing the user performance data to surgical simulation systems of one or more assessors, the one or more assessors assigned to review and evaluate the user performance data, receiving assessor input from the one or more assessors, and providing user feedback to the user based on the received assessor input. Each of the one or more surgical trainers, and the surgical simulation systems of the one or more assessors are at different locations.

In accordance with various embodiments of the present invention, a surgical simulation network is provided that has a surgical trainer with an external audio/video capture source that is positioned outside of the surgical trainer and an internal audio/video capture source that is positioned inside the surgical trainer. The external capture source is arranged to capture data from an area directly above the surgical trainer. The internal capture source is arranged to capture data of an area inside the surgical trainer. The surgical simulation network also has a processor that receives the captured data from the two sources, transmits the captured data to one or more assessor surgical simulation systems, receive assessor inputs, and provide feedback to the user.

In accordance with various embodiments of the present invention, a surgical simulation network is provided that has a surgical trainer that captures user performance data, one or more assessor devices that displays the captured user performance data and receives assessor input, and a surgical simulation system that receives the user performance data from the surgical trainer, transmits the user performance data to the assessor devices, receives the assessor inputs, and provides feedback to the user.

In accordance with various embodiments of the present invention, a surgical simulation network is provided that has a surgical trainer that captures user performance data related to a surgical simulation and a surgical simulation system that receives user performance data, processes the user performance data based on various guidelines, and automatically provides user feedback.

In accordance with various embodiments of the present invention, a surgical trainer is provided which has an external capture source positioned outside of the surgical trainer and pointed in a direction generally parallel with an upper surface of the surgical trainer, and an external capture source that is positioned inside the surgical trainer and arranged to be point in a direction transverse relative to the upper surface of the surgical trainer.

In accordance with various embodiments of the present invention, a surgical simulation network is provided that has a surgical trainer that has a surgical trainer with an internal and external capture source and a processor that receives and transmits the captured data.

In accordance with various embodiments of the present invention, a surgical simulation network is provided that has a surgical trainer that captures user performance data and a surgical simulation system that receives the performance data, transmits the performance data to one or more assessors, receives the inputs from the assessors, and provides user feedback based on the assessor inputs.

In accordance with various embodiments of the present invention, a surgical simulation network is provided that has a surgical trainer that captures user performance data and a surgical simulation system that receives the performance data, notifies the assessors of the availability of the performance data, transmits the performance data to the assessors requesting to review the data, receive the assessor inputs, and provide the user feedback based on the received assessor inputs.

In accordance with various embodiments of the present invention, a surgical simulation network is provided that has a surgical trainer that captures user performance data and a surgical simulation system that receives the performance data, transmits the performance data to one or more assessors, receives the assessors inputs, modifies the user performance data with the assessor inputs, and provides the modified performance data as feedback back to the user.

In accordance with various embodiments of the present invention, a surgical simulation network is provided that captures user performance data of the user performing various surgical simulations.

In accordance with various embodiments of the present invention, a surgical simulation network is provided that receives performance data, transmits performance data to one or more assessors, receives assessor inputs, and/or provides feedback to the user based on the assessor inputs.

Many of the attendant features of the present invention will be more readily appreciated as the same becomes better understood by reference to the foregoing and following description and considered in connection with the accompanying drawings.

### BRIEF DESCRIPTION OF THE DRAWINGS

The present inventions may be understood by references to the following description, taken in connection with the accompanying drawings in which the reference numerals designate like parts throughout the figures thereof.

FIG. 1 illustrates a surgical simulation network system in accordance with various embodiments of the present invention.

FIG. 2 illustrates a surgical trainer in accordance with various embodiments of the present invention.

FIG. 3 illustrates a surgical trainer with an associated computing device in accordance with various embodiments of the present invention.

FIG. 4 illustrates a surgical simulation system in accordance with various embodiments of the present invention.

FIG. 5 illustrate exemplary operations performed by a resource module of a surgical simulation system in accordance with various embodiments of the present invention.

FIG. 6 illustrate exemplary operations performed by a program schedule module of the surgical simulation system in accordance with various embodiments of the present invention.

FIG. 7 illustrate exemplary operations performed by a course module of a surgical simulation system in accordance with various embodiments of the present invention.

FIG. 8 illustrate exemplary operations performed by a video assessment module of a surgical simulation system in accordance with various embodiments of the present invention.

FIG. 9 illustrate exemplary operations performed by an assessment history module of a surgical simulation system in accordance with various embodiments of the present invention.

FIG. 10 illustrates an exemplary main view in accordance with various embodiments of the present invention.

FIG. 11 illustrates an exemplary course view in accordance with various embodiments of the present invention.

FIG. 12 illustrates an exemplary didactic view in accordance with various embodiments of the present invention.

FIG. 13 illustrates an exemplary cognitive assessment data view in accordance with various embodiments of the present invention.

FIG. 14 illustrates an exemplary reflection assessment data view in accordance with various embodiments of the present invention.

FIG. 15 illustrates an exemplary debrief data view in accordance with various embodiments of the present invention.

FIG. 16 illustrates an exemplary technical assessment data view in accordance with various embodiments of the present invention.

FIG. 17 illustrates an exemplary video assessment data view in accordance with various embodiments of the present invention.

FIG. 18 illustrates an exemplary view regarding selection/connection of A/V sources in accordance with various embodiments of the present invention.

FIG. 19A and FIG. 19B illustrates exemplary views relating to real-time assessments in accordance with various embodiments of the present invention.

FIG. 20 illustrates an exemplary assessment history view in accordance with various embodiments of the present invention.

FIG. 21 illustrates an artificial intelligence (AI) video assessment system in accordance with various embodiments of the present invention.

FIG. 22A illustrates a computer vision module of an AI video assessment system in accordance with various embodiments of the present invention. FIG. 22B illustrates a machine learning processor of an AI video assessment system in accordance with various embodiments of the present invention.

FIG. 23 illustrates exemplary operations performed by a video validation module of a surgical simulation system in accordance with various embodiments of the present invention.

FIG. 24 illustrates a computing system associated with a surgical simulation network system in accordance with various embodiments of the present invention.

### DETAILED DESCRIPTION

In accordance with various embodiments, a surgical simulation network system is provided, and various views of various embodiments of exemplary surgical simulation network systems and aspects thereof are shown in the figures. The surgical simulation network system facilitates the performance, review and assessment of a user's surgical skill and procedural aptitude through a remote or distributed environment. A surgical simulation system in conjunction with one or more surgical trainers allows users, such as trainees or residents, to simultaneously view and capture their simulated surgical performance directly from an internal audio/video input source within a surgical trainer as well as from an external audio/video input source outside of the surgical trainer. The surgical simulation system, in various embodiments, provides assessors, such as faculty or peers, to access and view the captured simulated surgical performances while simultaneously assessing them using assessment tools provided by the surgical simulation system. As used in this present disclosure, surgical simulation performance data (e.g., audio and/or video data) or recordings may comprise not only of captured surgical simulation performance data but may also include any number of pre-processing and/or post-processing steps (e.g., data cleaning/cleansing, integration, transformation, reduction) that can prepare the captured data suitable for use with the surgical simulation network system, e.g., formatting for compatibility and/or storage.

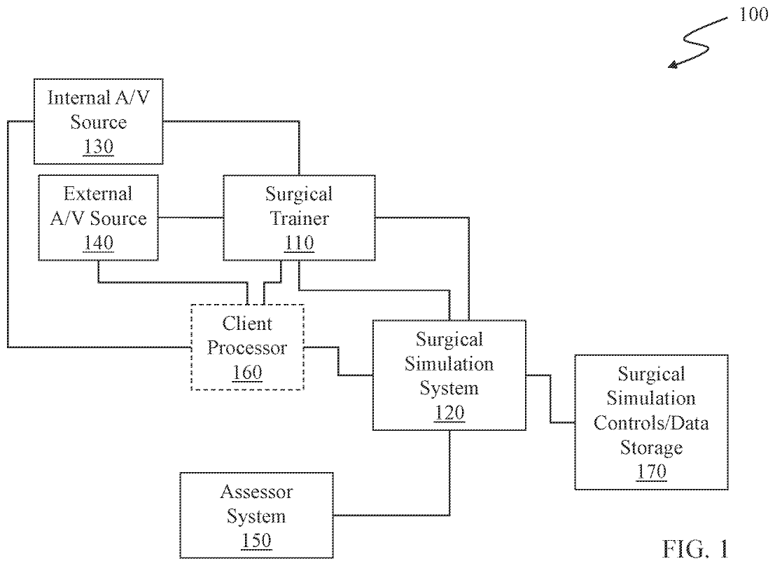

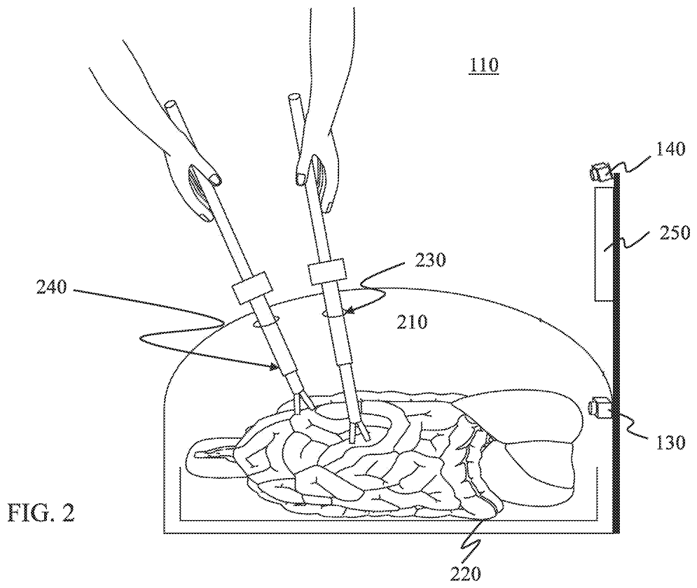

An exemplary surgical simulation network system 100 is illustrated in FIG. 1. Although the figure illustrates a particular arrangement of components making up the surgical simulation network system 100, it should be noted that other arrangements involving more, less, or different components are also possible and that one of ordinary skill in the art would appreciate and understand these possible different embodiments as being within the scope of the present disclosure.

The surgical simulation network system 100 comprises one or more surgical trainers 110 connected to one or more surgical simulation systems 120. The surgical trainers 110 are capable of receiving or using different surgical simulation models or exercises thereby allowing the overall surgical simulation network system 100 to be applicable to testing a variety of different surgical simulations. Attached to or otherwise provided with the one or more surgical trainers 110 are one or more audio and/or video input sources 130, 140 that are capable of capturing or recording user performance of a surgical simulation (hereinafter referred to as “A/V source”).

The users would use the surgical trainers 110 to perform surgical simulations. The user's performance can be captured and recorded at each surgical trainer 110 and subsequently sent to the simulation system 120 for storage, review, and/or assessment or evaluation. In some embodiments, the user's performance can be captured and sent (i.e. directly streamed) to the surgical simulation system 120 for storage, review, and/or assessment or evaluation. In some embodiments, the surgical trainers 110 can each be physically connected (i.e. wired connection) to the same surgical simulation system 120. However, in other embodiments, the location of different surgical trainers 110 can be scattered at different physical locations but each can still be connected to the same surgical simulation system 120 albeit through non-wired connections (e.g., Internet). This allows the surgical simulation network system 100 to span a variety of different locations (e.g., schools, hospitals) so that a vast number of users can practice a particular surgical simulation and be evaluated, e.g., via the same guidelines, as provided by the surgical simulation system 120.

As mentioned above, one or more surgical simulation systems 120 (which may be configured to be part of the same computer network) can be used by the overall surgical simulation network system 100 to review, assess and/or evaluate recordings of user's performance during a surgical simulation. In some embodiments, the surgical simulation system 120 may be remote or provided in a centralized facility remote from other surgical simulation systems 120 as well as from the surgical trainers 110. In various embodiments, the surgical simulation system 120 may be partly or entirely run in the cloud (i.e. processed and stored on the Internet). In various embodiments, there may be different surgical simulation systems 120 that can be used to evaluate user recordings based on, for example, the location of the user, the surgical trainers 110 being used, and/or the simulation being performed on the surgical trainers 110. The user's performance data can be automatically sent to one or more surgical simulation systems 120 based on a variety of different criteria. In various embodiments, each surgical simulation system 120 can be configured to still utilize the same guidelines to evaluate a user's performance in order to maintain uniformity for the entire surgical simulation network system 100. In other embodiments, different surgical simulation systems 120 may be used to capture, store, review, and assess or evaluate different surgical simulation scenarios and user's performances thereof and can have different guidelines as well. For example, a first surgical simulation system 120 can be devoted to a first surgical simulation while a second surgical simulation system 120 can be devoted to a different second surgical simulation. Furthermore, a first surgical simulation system 120 can be devoted to evaluating/assessing students while a second surgical simulation system 120 can be devoted to evaluating/assessing more advanced users (i.e. experts). Users can, in some embodiments, switch between different surgical simulations that can be practiced on using the same surgical trainer 110. Furthermore, in some embodiments, users may be capable of identifying which surgical simulation system 120 the user's performance data can be sent to.

In some embodiments, to facilitate the surgical trainer 110 in identifying what surgical simulation is being performed and captured by the user and where the captured simulation should be provided, each surgical trainer 110 can be provided with one or more client processors or computing devices 160. In various embodiments, the one or more surgical simulation systems 120 performs a variety of different operations such as identifying what surgical simulation is being performed, how the surgical simulation data is captured/recorded/uploaded from each of the surgical trainers 110, and where the captured simulation data should be transferred/stored. The client processors 160 can be configured to facilitate connections or communication between one or more surgical trainers 110 and the one or more surgical simulation systems 120. In various embodiments, one or more client processors 160 act as operational and/or communication intermediaries between 1) the one or more surgical trainers 110 and the internal and external A/V sources 130, 140, 2) the surgical simulation system 120 and the internal and external A/V sources 130, 140, or 3) one or more surgical trainers 110 and the surgical simulation system 120. The one or more client processors 160, in various embodiments, can be embedded or integrated into the one or more of the surgical trainers 110. Other embodiments allow the one or more client processors 160 to be associated computing devices (e.g., personal computer, laptop, smart devices) which can be connected to and communicate with the surgical trainer 110, one or more internal A/V sources 130 and/or one or more external A/V sources 140.

In some embodiments, the user can explicitly identify (e.g., via use of the client processor 160 such as selection from a drop-down menu) the surgical simulation that the user would like to perform. This identification can be used to inform the surgical simulation system 120, in various embodiments, to provide the appropriate surgical simulation template/program to the surgical trainer 110. Furthermore, the identification can also be used to facilitate informing the surgical simulation system 120 what type of data is captured/recorded/uploaded so that any additional processing and subsequent storage (e.g., identifying where the user performance data should be sent) can be performed accordingly. In some embodiments, the surgical trainer 110 may be capable of automatically identifying the surgical simulation being performed and automatically identify which surgical simulation system 120 to transfer the captured surgical simulation data. For example, based on analyzing and/or identifying organ models or training exercises being inserted within the surgical trainer 110, the surgical simulation system 120 can run the appropriate surgical simulation for the user at the respective surgical trainer 110 as well as prepare for the incoming data being captured/recorded for further processing and subsequent storage.

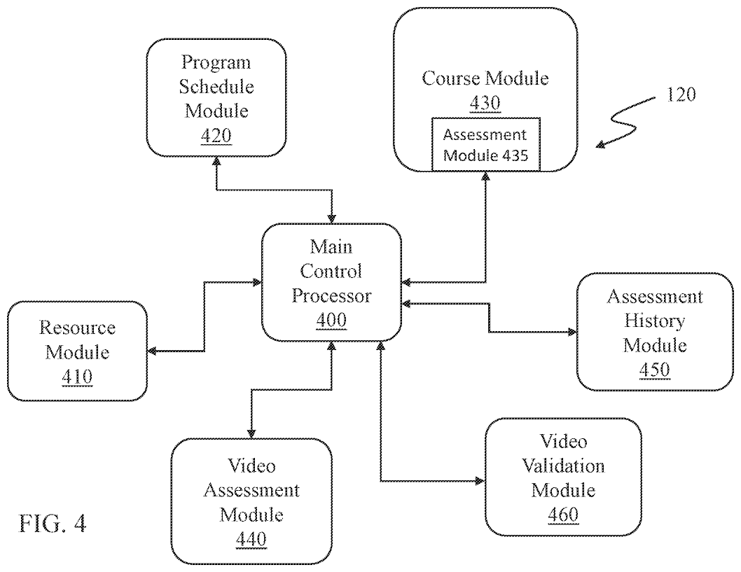

The surgical simulation system 120 comprises a video assessment module that facilitates the processing, review, and evaluation of a user's surgical skill in order to provide feedback (e.g., procedural aptitude, competence) back to the user. The feedback can be provided through a remote or distributed environment. Furthermore information related to the video assessment module is provided below (e.g., FIG. 4).

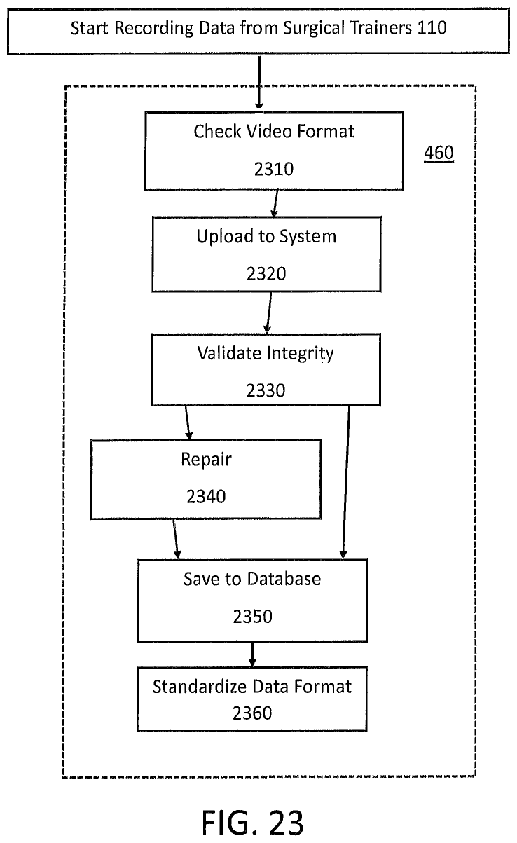

In various embodiments, the user performance data from the surgical trainers 110 may be directly transmitted (i.e. streamed) to the surgical simulation system 120. For these embodiments, the surgical simulation system 120 may be designed to have a video validation module that facilitates the recording and uploading of the user performance data. Furthermore, the video validation module would be configured to ensure that the user performance data has been properly received from the respective surgical trainer 110. In situations where the user performance data has not been properly received, the video validation module can attempt to “repair” the data or request the user to resubmit the same data again. Further information related to the video validation module will be provided below (e.g., FIG. 23).

The surgical simulation system 120 in conjunction with the surgical trainer 110 allows users, such as trainees and residents, to simultaneously capture and view their simulated surgical performance directly from one or more internal audio/video input sources 130 associated with the surgical trainer 110. For example, digital recording devices (e.g., camera) and/or sensors can be placed within the interior or cavity of the surgical trainer 110 that are designed to obtain and capture user performance of a surgical simulation. Such user performance may include (but would not be limited to) the tracking of movement of the surgical devices (e.g., probes, graspers) being used and where/how the surgical devices interact with the model enclosed within the surgical trainer 110. In various embodiments, the internal audio/video input sources 130 are pointed in a direction transverse relative to an upper surface of the surgical trainer. In various embodiments, the audio/video input sources 130 can be adjusted (i.e. tilted) to cover different areas within the internal cavity of the surgical trainer. In various embodiments, the ability to adjust (i.e. tilt) the input source is based on the flexibility of the input sources (e.g., no larger than 45 degrees). In various embodiments, the internal audio/video input sources 130 are inserted through the upper surface of the surgical trainer so that they are positioned to capture objects and activity within the interior of the surgical trainer. Thus, in various embodiments, the internal audio/video input sources 130 can be removable and replaceable with other audio/video input sources. In various embodiments, the internal audio/video input sources 130 may be manufactured/produced separately from the surgical trainer, inserted through an upper surface of the surgical trainer and affixed to a particular location so as to prevent removal from the surgical trainer.

The user performance data being captured by the audio/video input sources are then subsequently provided to the surgical simulation system 120. In some embodiments, digital recording devices can also be attached to user manipulable devices (e.g., probes, graspers) which are inserted into the surgical trainer 110 and provide a recording of the user's maneuvers and actions within the surgical trainer 110.

Recordings can also include external audio/video input sources 140 outside of the surgical trainer 140. The external audio/video input sources 140 can be configured to capture data associated with the user (e.g., the user's body posture and hand movements) and/or the outer portions of the surgical trainer 110. In various embodiments, the external audio/video input sources 140 are arranged to be pointed in a direction generally parallel with an upper surface (associated with the upper cover) of the surgical trainer in order to capture data associated with an area above the surgical trainer (i.e. the user's hands and their control of surgical devices being inserted through the upper cover). In various embodiments, the external audio/video input sources 140 are positioned at a distal portion of the surgical trainer. Similarly, such data can be then provided to the surgical simulation system 120.

In various embodiments, both the internal and external input sources 130, 140 are used simultaneously. As such, one or both of the input sources 130, 140 may be fixed or movable. In various embodiments, one input source can be fixed while the other input source is movable. IN all cases, the data obtained from the internal and external A/V input sources 130, 140 can be combined or separated based on how the surgical simulation system 120 is configured to use the respective data to quantify/assess a user's performance.

The surgical simulation system 120, in various embodiments, can have an associated assessor system 150. The assessor system 150 allows persons independent of the user, such as faculty or peers, to access and view a recorded surgical simulation performance in order evaluate/assess the user's aptitude/competency. The assessor system 150 may require assessors to access the recorded surgical simulation performance via pre-defined means (e.g., user accounts, password) to control who can review and evaluate which specific recorded surgical simulation. The assessor system 150 may also be designed to limit which assessors can access which recorded surgical simulation data (i.e. allowing assessors to evaluate performance of peers not located within the same class or school). Furthermore, the assessor system 150, the surgical simulation system 120 or both, can also provide various assessment tools that would facilitate in the review and evaluation of any recorded surgical simulation data associated with the surgical simulation network system 100. For example, the assessor system 150, the surgical simulation system 120 or both, could provide data for various points of interest in the recording (e.g., elapse time, path taken). The assessor system 150, the surgical simulation system 120 or both, could also provide a rubric or other types of guidelines (e.g., benchmarks) that would also facilitate the different assessors in providing a uniform way for reviewing and evaluating the recorded surgical simulations. For example, a sample simulation can be shown illustrating how a particular surgical procedure should be performed. Furthermore, different guidelines can be provided based on the experience (e.g., resident, practicing doctor) on the user. In various embodiments, stricter benchmarks would be in place for more experienced users compared to students who are learning how to perform a particular simulated surgical procedure. In some embodiments, users may be allowed to assess their own performance via a self-assessment option. Similarly, the users performing self-assessment can select and use the various different guidelines available in order to determine how they would've been characterized.

Further details related to the surgical trainer 110 will be provided as follows. As illustrated in FIG. 2, an exemplary surgical trainer 110 is illustrated. In various embodiments, the surgical trainer 110 provides an internal cavity 210 substantially obscured from the user which simulates the internal space that a surgeon could operate in during a surgical procedure for a patient. The internal cavity 210 is configured to receive one or more simulated or live tissues, organ models, or training exercises 220. The tissues, models, or training exercises 220 can be used to simulate different surgical procedures and conditions. The users would then be able to practice various surgical procedures. The internal cavity 210, in various embodiments, is accessed via a tissue simulation region 230 associated with an outer surface of the surgical trainer 110 that is penetrable by the user employing devices 240 (e.g., graspers) to practice surgical techniques on the tissue, model, or exercise 220 located within the internal cavity 210. Additional or embedded trocars, cannulas, seals, hand-assisted access devices, single-site port devices or the like may also be used to access the internal cavity 210 through the tissue simulation region 230. Additionally, trocars, cannulas, seals, hand-assisted access devices, single-site port devices or the like may also be used or provided in order to access the internal cavity 210 which are embedded or attached to an upper portion of the surgical trainer 110 to access the internal cavity 210 in lieu of a tissue simulation region 230 (or in other embodiments in addition to the tissue simulation region 230). The surgical trainer 110 in various embodiments is particularly well suited for practicing laparoscopic or other minimally invasive surgical procedures although other surgical procedures are also possible in other embodiments.

In various embodiments, the surgical trainer 110 comprising an upper cover and a base positioned below or under the upper cover. Disposed between the upper cover and the base is the internal cavity 210. The surgical trainer 110, in various embodiments, is configured to mimic the torso of a patient such as the abdominal region. The upper cover is representative of the anterior surface of the patient and the space between the upper cover and the base (i.e. internal cavity 210) is representative of an interior of the patient or body cavity where organs reside. The surgical trainer 110 is configured for teaching, practicing, and/or demonstrating various surgical procedures and their related instruments in simulation of a patient undergoing a surgical procedure. Surgical instruments (e.g., devices 240) are inserted into the internal cavity 210 through the tissue simulation region 230 and/or through pre-established apertures or ports in the upper cover of the surgical trainer 110.

As discussed above in FIG. 1, the surgical trainer 210 may include internal A/V sources 130 and/or external A/V sources 140 which would be used to capture portions of the user (e.g., hands) and/or the user's performance during the simulated surgical procedure. The viewing of A/V data or recording obtained from the internal and/or external A/V sources 130, 140 may be provided for the user (and other onlookers) to view in real-time on a display screen 250 positioned above the upper cover of the surgical trainer 110. The user may also request a playback of captured user performance data previously obtained at a later time as well.

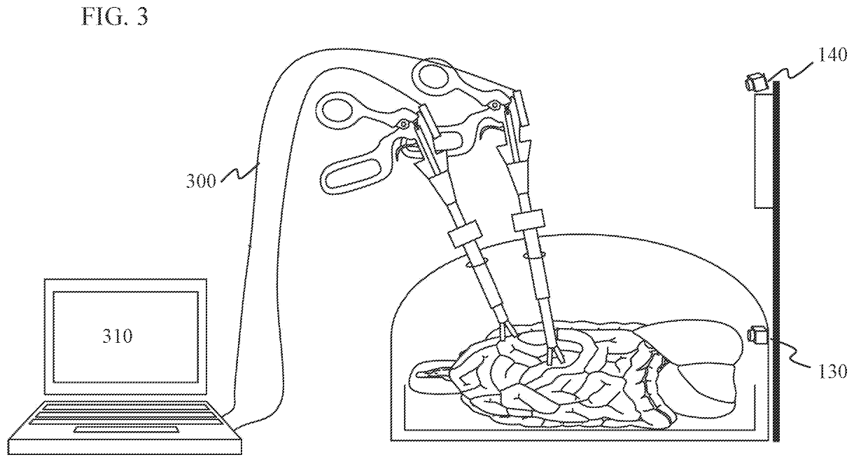

In various embodiments, the surgical trainer 110 comprises a video display or monitor 250. The video display 250 is connectable to a variety of visual systems for delivering an image to the video display 250. For example, a laparoscope inserted through one of the pre-established ports or via the tissue simulation region 230 and/or an internal video device 130 (e.g., camera) located in the internal cavity 210 of the surgical trainer 110 may be used to observe the simulated procedure within the surgical trainer 110 and may be connected to the video display 250. In some embodiments the captured data of the surgical procedure can also be provided to a separate computing device 310 such as a personal computer, laptop, or smart device as illustrated in FIG. 3. The data obtained from the devices 240 (e.g., laparoscope) can be transmitted to the computing device 310 via a wired connection 300, however, non-wired transmission is also possible. In some embodiments, the devices 240 may be capable of capturing the simulated surgical procedure and store the captured data into memory (e.g., USB) so that at a later time the captured A/V data can be transmitted/transferred to the computing device 310 as well as the surgical simulation system 120. As noted above, the captured data can also be directly provided (i.e. streamed) to a surgical simulation system 120.

As noted above, a first internal A/V source 130, e.g., camera, laparoscope or endoscope, is provided to view and capture user performance data associated with the internal cavity 210 or contents thereof, such as distal ends of inserted devices 240 or instruments (e.g., graspers, laparoscope), that is substantially obscured by the surgical trainer 110. In various embodiments, a second or different outer A/V source 140, e.g., camera, is provided to view and capture A/V data associated with the outer portions of the surgical trainer 210 and in particular proximal ends of inserted device 240 or instruments near or adjacent to the tissue simulation region 230 and/or the upper cover. The second or different outer A/V source 140 can also be configured to capture data associated with at least a portion of the user (e.g., hands) also during the surgical simulation. In various embodiments, one or more outer A/V sources 140 are provided, arranged or configured to specifically capture and/or record one or more views of the outer portions of the surgical trainer 110 and in particular proximal ends of inserted instruments and/or the user (e.g., hands) near or adjacent to the tissue simulation region 230 and/or the upper cover that may be otherwise obscured by the user. In various embodiments, one or more inner A/V sources 140 are provided, arranged, or configured to specifically capture and/or record one or more views of the internal cavity 210 or contents thereof, such as distal ends of inserted devices or instruments 240 and/or the simulated model or exercise 220, that is substantially obscured by the surgical trainer 110 from the user. The inner A/V sources 140 can also be provided, arranged, or configured to capture and/or record portions and/or contents within the surgical trainer, including the distal ends of inserted devices or instruments 240, that may be obscured by other inserted instruments and/or the simulated surgical model or exercise 220 contained within the surgical trainer 110. In some embodiments, the first internal and/or second external A/V sources 130, 140 may be integrated with the surgical trainer 110. In some embodiments, the first internal and/or the second external A/V sources 130, 140 may be arranged or configured to provide only video capabilities or audio capabilities (e.g., capture only video or capture only audio during a surgical simulation).

In various embodiments, the surgical simulation network system 100 may be capable of using one or more internal A/V sources without the associated one or more external A/V sources and vice versa. For example, the surgical simulation system may only require data from the internal A/V sources. In some embodiments, assessors may request to view only data coming from the internal A/V sources. In various embodiments, the surgical simulation network system 100 may be configured to either capture only the A/V data being required/requested and/or extract the requested/required A/V data and process and/or present only that specific data. The same is also possible for embodiments where only the external A/V sources are required by the surgical simulation system and/or requested by assessors.

In various embodiments, the one or more surgical trainers 110 communicate and/or supply the audio and/or video data from the one or more internal or external A/V sources 130, 140 directly to the surgical simulation system 120. In various embodiments, one or more client processors 160 connected to the internal and/or external A/V sources 130, 140 and/or surgical trainers 110 communicate or supply the audio and/or video data from the A/V sources 130, 140 to the surgical simulation system 120. In various embodiments, a portable memory storage device, such as a flash drive, a computing device, a smart phone, or other digital mobile device may also be provided and connected to the one or more surgical trainers 110, to receive the captured A/V data associated with the simulated surgical performances and assist in the playback of any recorded videos on the display of the computing device (e.g., laptop) 310 or the display 250 associated with the surgical trainer 110. In various embodiments, the surgical trainer 110 comprises one or more connections or ports to connect to one or more client processors 160 (e.g., a laptop or desktop computer, a mobile digital device or tablet) via wires or wirelessly (e.g., internet). In various embodiments, one or more connections of the surgical trainer 110 are provided to deliver video and/or audio output to a display that is different (e.g., larger than) from the video display 250 of the surgical trainer 110.

In various embodiments, the upper cover and the base of the surgical trainer 110 are substantially the same shape and size and have substantially the same peripheral outline. In various embodiments, the internal cavity 210 of the surgical trainer 110 is partially or entirely obscured from the user's view. In other words, the user is incapable of directly viewing, for example, the simulated model or exercise 220 contained within the surgical trainer 110. In various embodiments, one or more legs or side walls are provided to separate and/or angle the upper cover from or relative to the base. In some embodiments, the top cover may obscure the view of the user from the top of the surgical trainer 110. However, the side walls and/or legs may provide one or more openings on the side of the surgical trainer 110 which may allow the user and/or other viewers to view the interior of the surgical trainer 110.

In various embodiments, one or more simulated tissues, organ models or training exercises 220 are provided for the surgical trainer 110. These simulated models or training exercises 220 are removable from the surgical trainer 110 and interchangeable with other tissues, models, or training exercises 220. In various embodiments, the one or more simulated tissues, organ models, or training exercises may be made of one or more organic base polymer including but not limited to hydrogel, single-polymer hydrogel, multi-polymer hydrogel, rubber, latex, nitrile, protein, gelatin, collagen, soy, non-organic base polymer such as thermo plastic elastomer, KRATON, silicone, foam, silicone-based foam, urethane-based foam and ethylene vinyl acetate foam and the like. Into any base polymer one or more filler may be employed such as a fabric, woven or non-woven fiber, polyester, nylon, cotton and silk, conductive filler material such as graphite, platinum, silver, gold, copper, miscellaneous additives, gels, oil, cornstarch, glass, dolomite, carbonate mineral, alcohol, deadener, silicone oil, pigment, foam, poloxamer, collagen, gelatin and the like. The adhesives employed in such models or exercises may include but are not limited to cyanoacrylate, silicone, epoxy, spray adhesive, rubber adhesive and the like.

The different tissues, models, or training exercises 220 may each be designed to simulate different surgical procedures or allow for the training of different skillsets involved in the different surgical procedures. Furthermore, aspects of the different simulated models or training exercises 220 may also be customizable. The customization of the tissues, models, or training exercises 220 may allow for adjusting difficulty in accordance to the user performing the simulated surgical procedure.

Returning to FIG. 1, in various embodiments, one or more assessor systems 150 may be provided for the surgical simulation network system 100. The one or more assessor systems 150 are capable of accessing audio and/or video data for the surgical simulation systems 120 as supplied by the internal and external A/V sources 130, 140. The assessor system 150 can also receive or access assessment data, tools and/or forms from the surgical simulation system 120. In various embodiments, the one or more assessor systems 150 provides access to an assessor, such as a faculty member, surgical expert, or peer, and the ability to view the audio and/or video data (e.g., the recordings of the user performing the surgical simulation) in conjunction with supplied assessment data, tools and/or forms to evaluate the surgical performance as presented by the audio and/or video data of a particular user (e.g., surgical trainee).

In accordance with various embodiments, the surgical simulation system 120 is coupled to one or more storage systems or repositories for holding or storing surgical simulation controls and/or data 170. The surgical simulation control and/or data storage 170, in various embodiments, may comprise of one or more internal or remote databases arranged to store or hold data or informational/operational details utilized by the surgical simulation system 120 (or for the overall surgical simulation network system 100 as a whole). The data or informational/operational details may be used to populate or present various views (e.g., passive and/or actionable views) that are presentable by the surgical simulation system 120. Furthermore, the surgical simulation control and/or data storage 170 may also include information that would be useable to facilitate in the user manipulation of the different views. Such views are provided by the surgical simulation system 120 and communicated to the one or more surgical trainers 110, and/or one or more assessor systems 150. In various embodiments, such views are also capable of being provided by the surgical simulation system 120 and communicated to the one or more surgical trainers 110 and/or one or more assessor systems 150 via one or more client processors 160 acting as intermediaries between the surgical trainers 110 and/or surgical simulation/assessor systems 120, 150. In various embodiments, the surgical simulation data storage 170 comprises the audio and/or video data provided by the one or more internal and/or external A/V sources 130, 140 associated with the surgical trainer 110.

Referring now to FIG. 4, further details will be provided regarding various embodiments of the surgical simulation system 120. In various embodiments, the surgical simulation system 120 comprises a main control processor 400 connected to or integrated with one or more modules, sub-systems and/or sub-processors. In the figure, exemplary modules include a resource module 410, a program schedule module 420, a course module 430, a video assessment module 440 and/or an assessment history module 450. In embodiments where user performance data is transferred directly (i.e. streamed) from the surgical trainer 110 to the surgical simulation system 120, a video validation module 460 may also be included. The main control processor 400 coordinates the operation and communication between the different modules, sub-systems, and/or sub-processors to carry out the functionality of the surgical simulation system 120. In various embodiments, the main control processor 400 can be configured to retrieve, store, and validate operational, control and/or informational data into the surgical simulation controls/data storage 170. Further details regarding the various modules illustrated in FIG. 4 will be provided below.

The resource module 410 comprises, for example, general resource data and course activity data. The general resource data includes but is not limited to program expectations, course materials, and setup information that can be used to configure the surgical trainers for the different surgical simulations that users can perform. The course activity data in various embodiments includes but is not limited to course activity guide data.

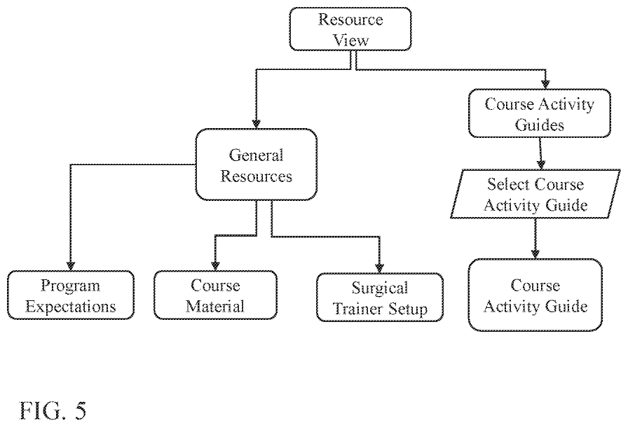

Exemplary operations performed by the resource module 410 are provided in FIG. 5. In various embodiments, the resource module 410 presents one or more resource views for the surgical simulation system 120 to retrieve and in turn provide resource details and/course activity guides for a user to view, such as a surgical trainee, on the surgical trainer 110 or associated computing device.

In various embodiments, the general resource data and course activity data can be stored with the surgical simulation system 120. In some embodiments, part or all of the general resource data and course activity data can also be stored in the surgical simulation controls/data storage 170 which would be made accessible directly by the resource module 410 and/or through the main control processor 400.

The program schedule module 420 of the surgical simulation system 120 comprises event card data and overall lab data. Such data includes but is not limited to lab event cards and resident progress data. Similar to the data associated with the resource module 410, the data for the program schedule module 420 can be stored with the surgical simulation system 120 but also (in part or entirely) stored in the surgical simulation controls/data storage 170. If the event card data and overall lab data for the program schedule module 420 is stored in the surgical simulation storage 170, such data would be accessible directly by the program schedule module 420 and/or through the main control processor 400.

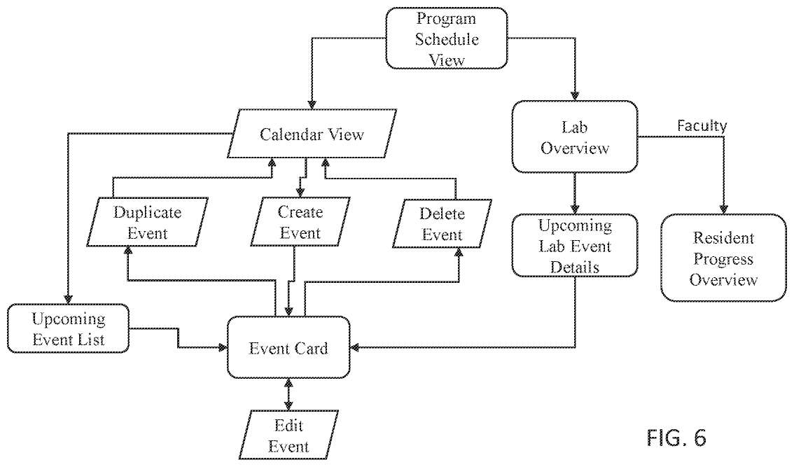

The exemplary operations performed by the program schedule module 420 are provided in FIG. 6. In particular, the program schedule module 420 is configured to modify, remove, or duplicate portions of the event card data. As such, in various embodiments, the program schedule module 420 presents one or more program schedule views, calendar views and/or lab overview views for the surgical simulation system for a user, such as a surgical trainee.

The course module 430 comprises course data including but not limited to curriculum outline data, course activity guide data, and didactic data. Such data can be stored in the surgical simulation system 120 but also in the surgical simulation controls/data storage 170. The course module 430, in some embodiments, can also include an assessment module 435. The assessment module 435 comprises cognitive assessment data, reflection assessment data, technical assessment data, and debrief assessment data and is configured to compile the cognitive assessment data, reflection assessment data, technical assessment data and debrief assessment data. Such data, in various embodiments, can be stored in the surgical simulation system 120 but also in the surgical simulation controls/data storage 170. In both cases (with the course module 430 and the associated assessment module 435), the data stored in the surgical simulation controls/data storage 170 can be accessible directly by the course module 430/assessment module 435, respectively and/or through the main control processor 400.

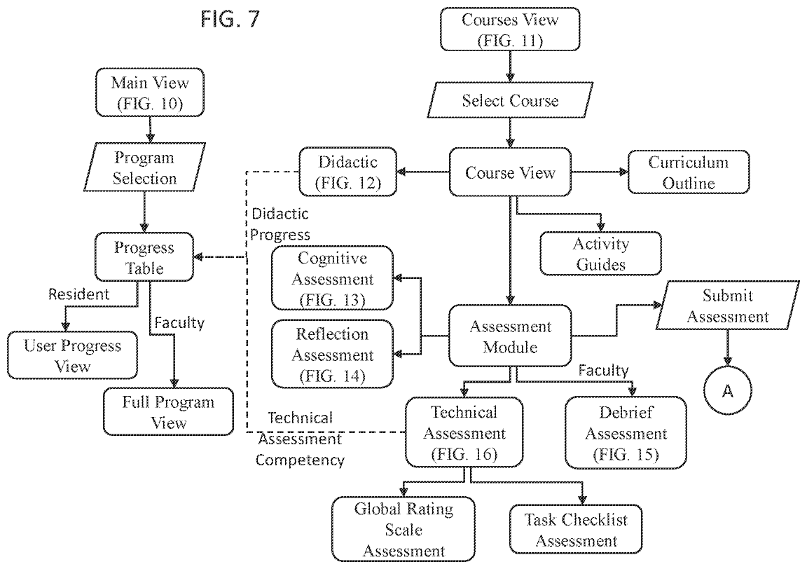

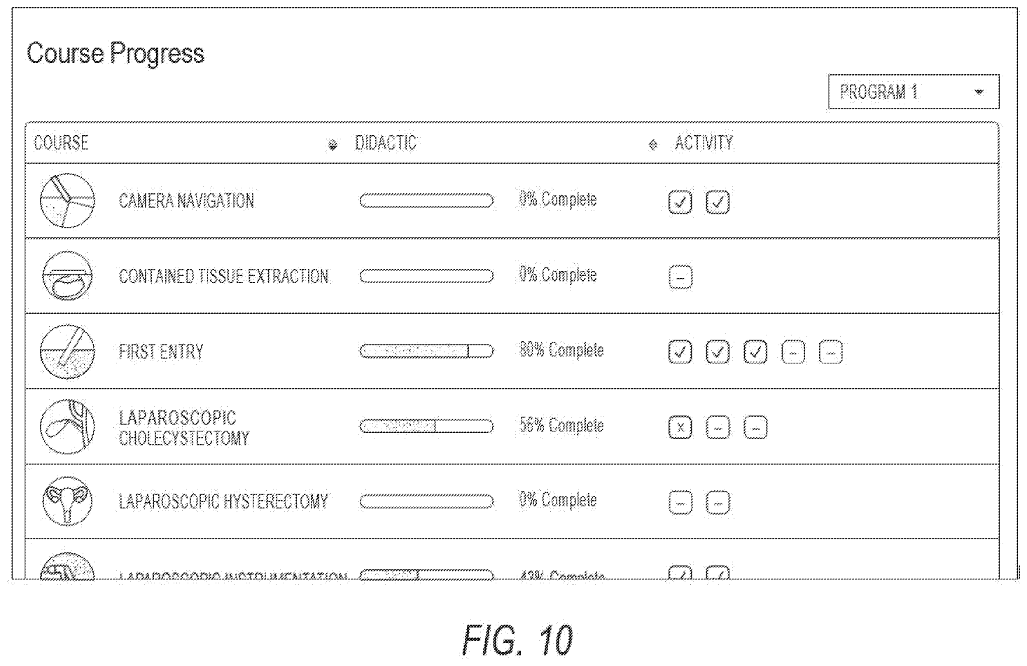

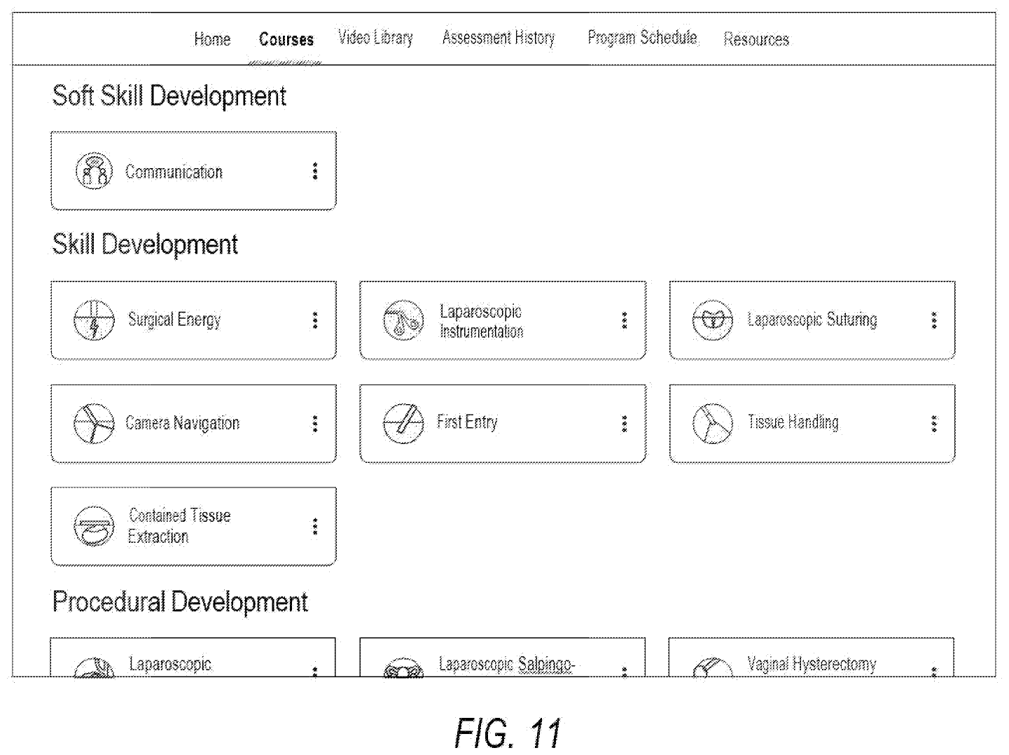

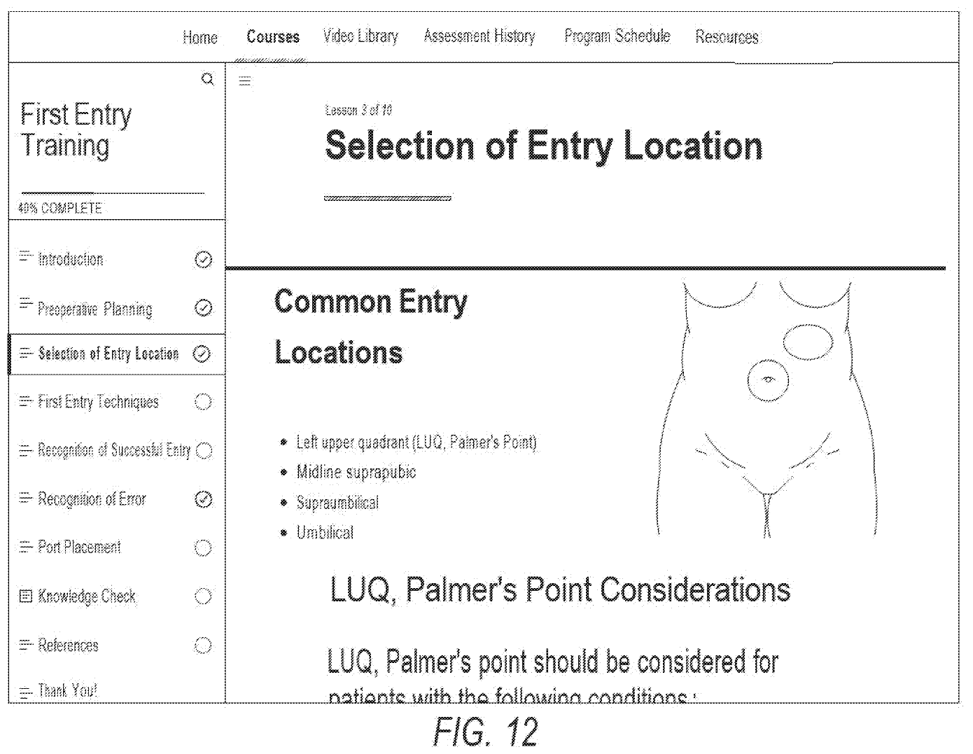

Exemplary operations performed by the course module 430 (including the associated assessment module 435) are provided in FIG. 7. The course module 430, the associated assessment module 435 and/or the main control processor 400 can be configured to present main views (e.g., FIG. 10), one or more course views (e.g., FIG. 11), and/or one or more didactic views (e.g., FIG. 12) for the surgical simulation system 120 for a user, such as a surgical trainee. The different views (as illustrated in FIG. 10-12) can be shown on the same display 250 associated with the surgical trainer 110 or on an associated computing device (such as a laptop) that is connected to the surgical trainer and/or the surgical simulation system 120. In various embodiments, different views can be provided simultaneously on a combination of multiple devices and/or displays.

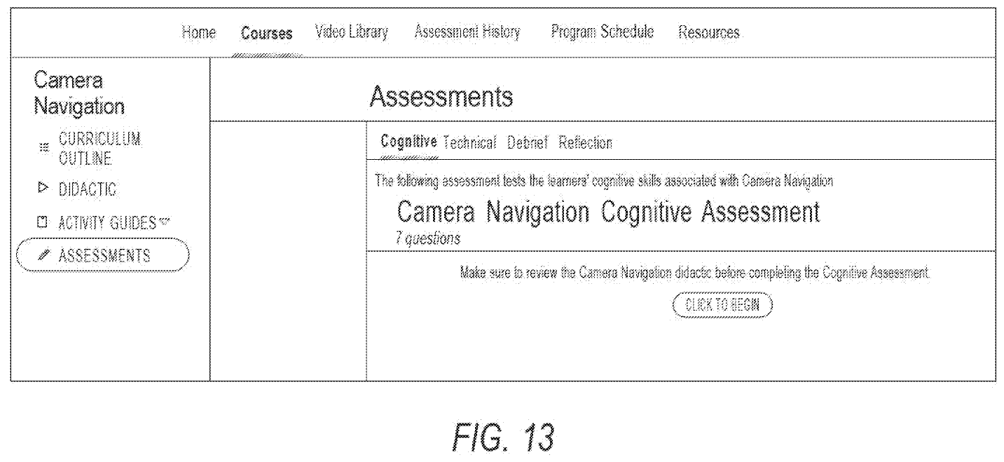

In various embodiments, the cognitive assessment data of the course module 430 comprises one or more views or tools with selectable pre-populated data or user populated data concerning or testing course retention or retention of surgical details and/or skills associated with a pre-determined or pre-selected simulated surgical procedure or exercise. One such exemplary view for the cognitive assessment data is shown in FIG. 13. In various embodiments, the cognitive assessment data comprises a collection of a surgical trainees' data provided by the surgical trainee after completion of predetermined didactic material associated with a particular course that tests retention of the course material.

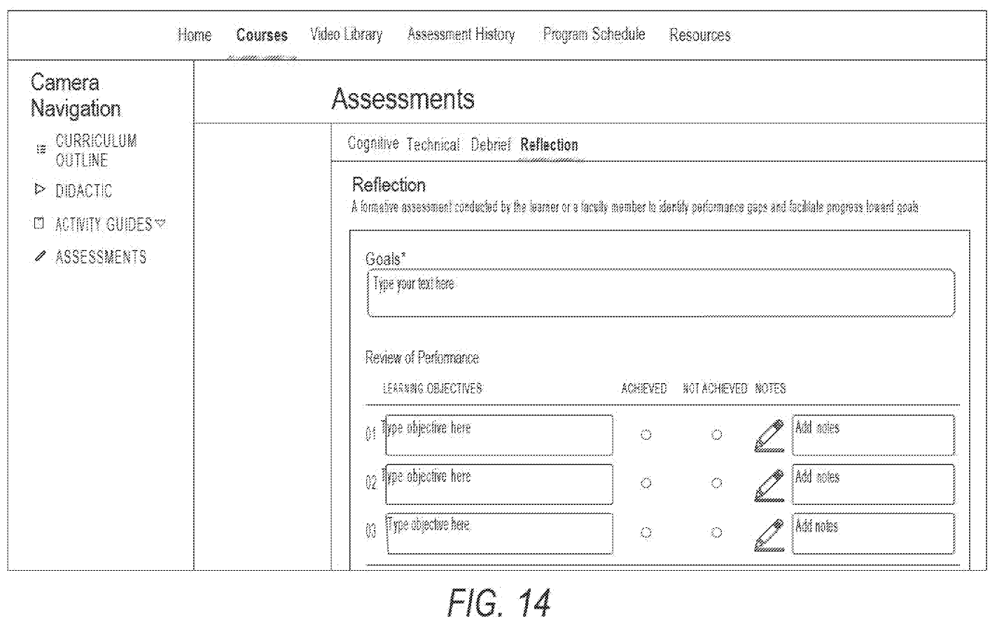

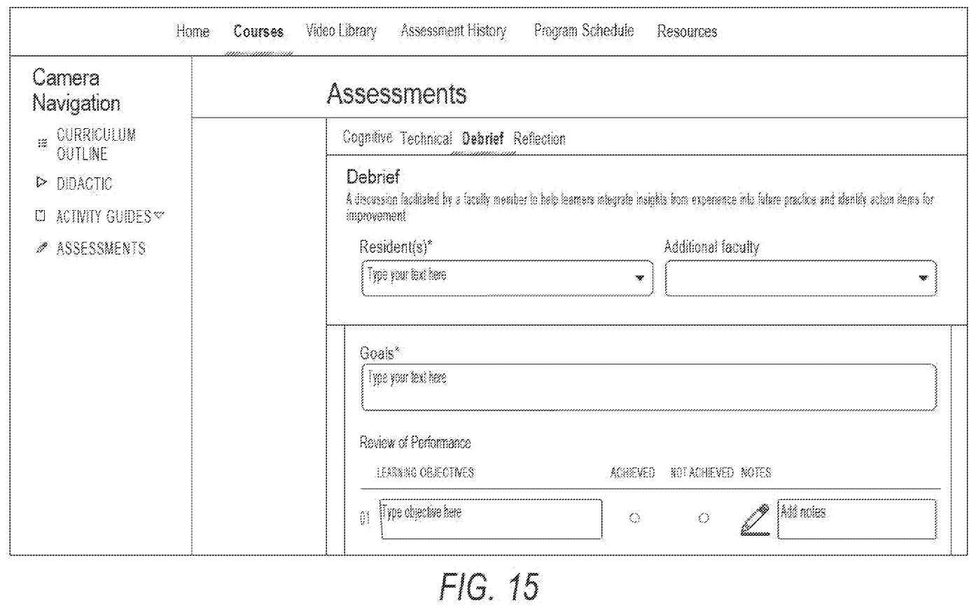

In various embodiments, the reflection assessment data and/or the debrief data associated with the course module 430 and/or assessment module 435 comprises one or more views or tools with pre-populated and/or user (e.g., a resident) and/or an assessor (e.g., faculty) populated data or selectable data concerning annotations, identifiers, or notes associated with a completed pre-determined or pre-selected simulated surgical procedure or exercise. In various embodiments, the reflection assessment data comprises a collection of a surgical trainees' data and/or faculty data provided by the respective surgical trainee and/or faculty identifying performance gaps and goal progression. Furthermore, in various embodiments, the debrief assessment data comprises a collection of a surgical trainees' data and/or faculty data provided by the surgical trainee and/or faculty identifying future practice insights and improvement action items. Such exemplary views associated with the debrief data and/or reflection assessment data are shown in FIG. 14 and FIG. 15.

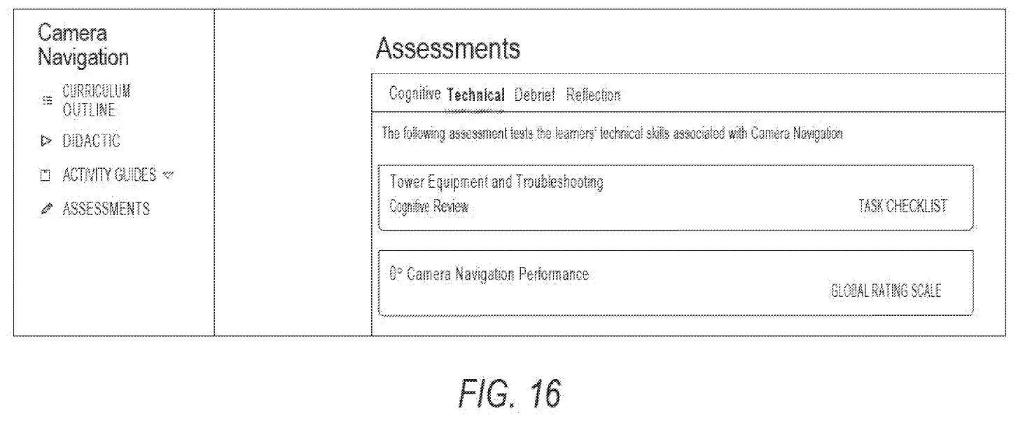

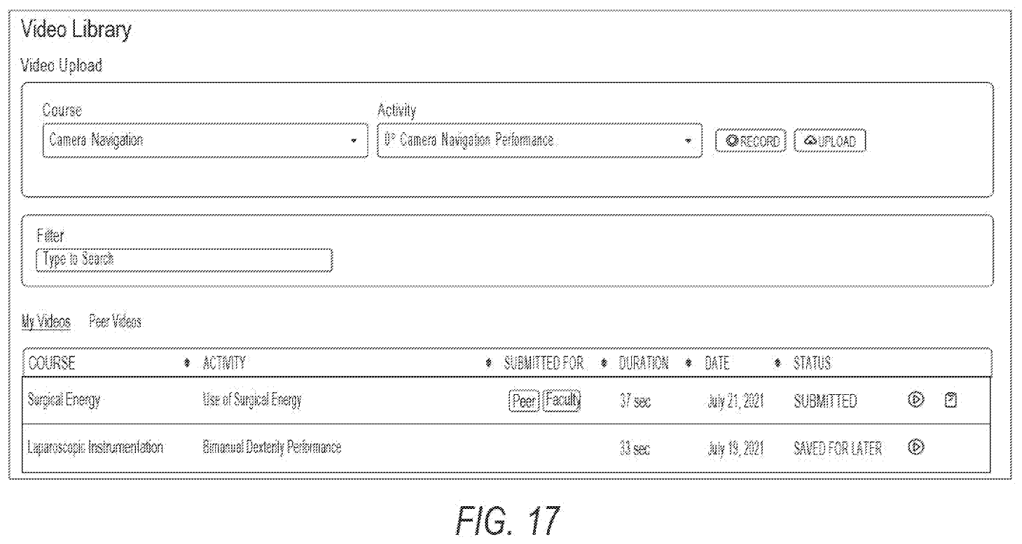

In various embodiments, the technical assessment data associated with the course module 430 and/or assessment module 435 comprises one or more views or tools with pre-populated and/or user populated or selectable data concerning or assessing a surgical trainee's technical skills associated with a pre-determined or pre-selected simulated surgical procedure or exercise. The technical assessment data, in various embodiments, may be tagged, linked, or otherwise associated with one or more surgical video performances. One such exemplary view is shown in FIG. 16.

The course module 430 in conjunction with the associated assessment module 435 are configured to determine didactic progress and/or technical assessment competency. In various embodiments, using the determined didactic progress and the determined technical assessment competency, program process for each user is determined. Didactic progress in various embodiments provides or identifies each user's completion identifier, status, or percentage of a particular course and technical assessment competency. In various embodiments, didactic progress also is capable of providing a competency identifier, status, or score of a surgical trainee of a particular course or simulated surgical procedure, exercise or performance.

The video assessment module 440 facilitates the performance, review, and assessment of surgical skill and procedural exercises of the user (e.g., resident, trainee) in a remote or distributed environment. The video assessment module 440 comprises surgical multimedia data (e.g., surgical performances, course data, and activity data). In various embodiments, such data is stored in the surgical simulation controls/data storage 170 and/or with the surgical simulation system 120. If the surgical multimedia data is stored in the surgical simulation controls/data storage 170, such data can be made accessible directly by the video assessment module 440 and/or through the main control processor 400.

The surgical multimedia data of the video assessment module 440 includes but is not limited to audio and/or video data from the one or more internal and/or external audio and/or video input sources 130, 140 associated with the surgical trainer 110. The video assessment module 440 is configured to receive, store, remove, retrieve, and display the surgical multimedia data. The video assessment module 440, in various embodiments, is configured to tag, link, or otherwise associate course data and/or activity data to the surgical multimedia data. In various embodiments, the video assessment module 440 can be configured to timestamp the surgical multimedia data and determine the duration of the surgical multimedia data. In various embodiments, assessors (via the video assessment module 440) are able to incorporate additional information (i.e. tags, links) that could inform the user how to better perform a surgical simulation.

The video assessment module 440, in various embodiments, can also comprise self-assessment data, peer assessment data, and faculty assessment data. The video assessment module 440 is configured to tag, link, or otherwise associate the self-assessment data, peer assessment data, and faculty assessment data to the selected surgical multimedia data by the video assessment module 440 with or without user interaction (e.g., automatically). In various embodiments, the assessment-related data and/or the links between the assessment-related data and the selected surgical multimedia data can be stored in the surgical simulation controls/data storage 170 and be accessible directly by the video assessment module 440 and/or through the main control processor 400. Alternatively, the assessment-related data and/or the links between the assessment-related data and the selected surgical multimedia data can be stored with the surgical simulation system 120. In this way, the users can later access the user performance data and view with the updated tags, links, or other data associated with the user performance data provided by the assessors. The tags, links, or other data can be used as a form of feedback by the users.

In various embodiments, manual tagging or linking of the assessment-related data and the selected surgical multimedia can comprise an assessor identifying particular frames or portions of a frame and associating the assessment-related data accordingly. The assessment-related data could comprise identifying locations of points of interest (e.g., organs), objects of interest (e.g., surgical devices), and interactions between the user and the points of interest. In various embodiments, the computing device may be configured to perform the labeling, for example, via the use of computer-vision which can identify the various points of interest and/or objects of interest automatically. The tags/links can then be used as reference when assessors review the user performance data and provide a corresponding assessment.

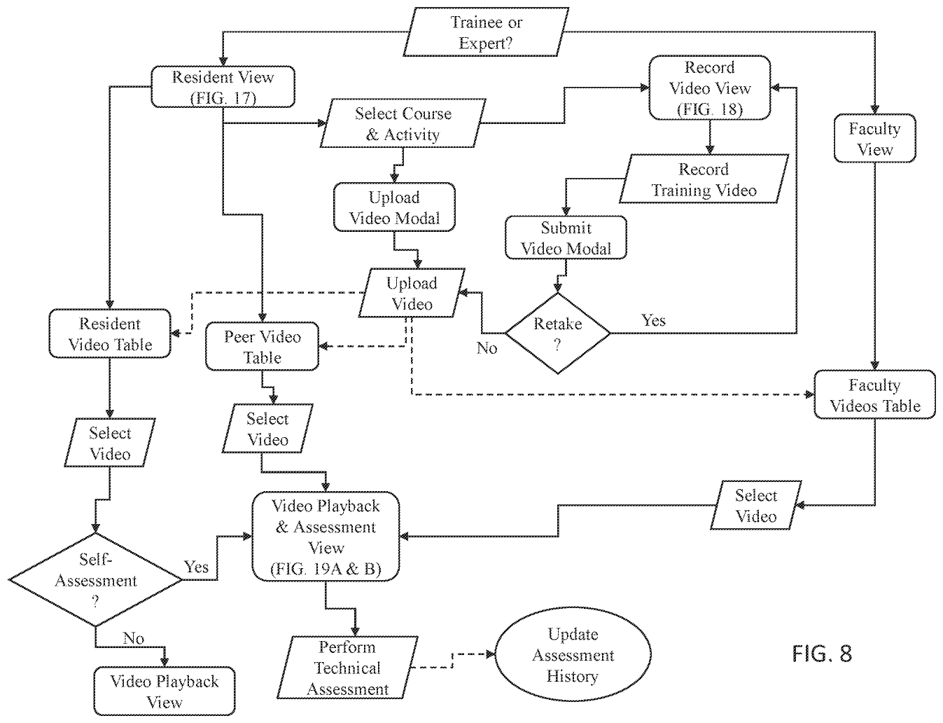

In various embodiments, the video assessment module 440 is configured to provide one or more views or tools with selectable pre-populated and/or user populated data concerning a user's surgical performance. In other words, assessors may be directed on selecting various pre-populated choices (i.e. good/bad) used to characterize a user's performance being reviewed, may be directed to input their own comments explaining and quantifying the user's performance, or a combination of both. Exemplary operations performed by the video assessment module 440 are provided in FIG. 8. These operations include identifying the experience of the user, selection of the surgical simulation being performed, providing the user performance data to others for viewing in order to review/assess the user's performance, and saving the data and assessments on the network so that the user can access at a later date/time. Furthermore, an exemplary view provided by the video assessment module 440 can be seen in FIG. 17. In particular, the figure illustrates an interface where the user is able to identify a surgical simulation being performed to be recorded and uploaded. Furthermore, the user is able to view other uploaded videos that were previously recorded by the user.

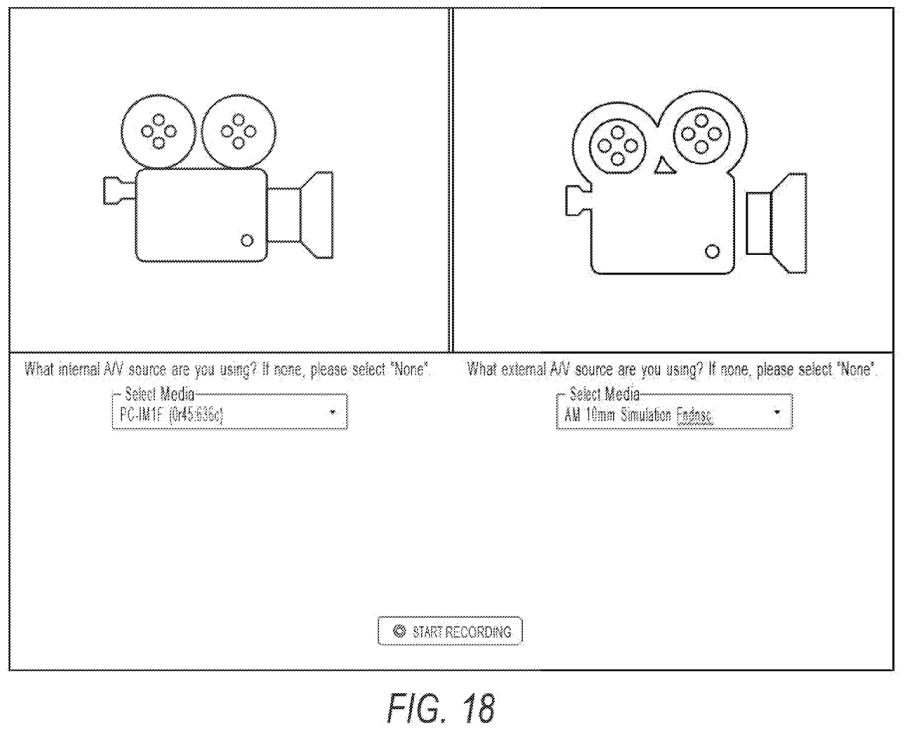

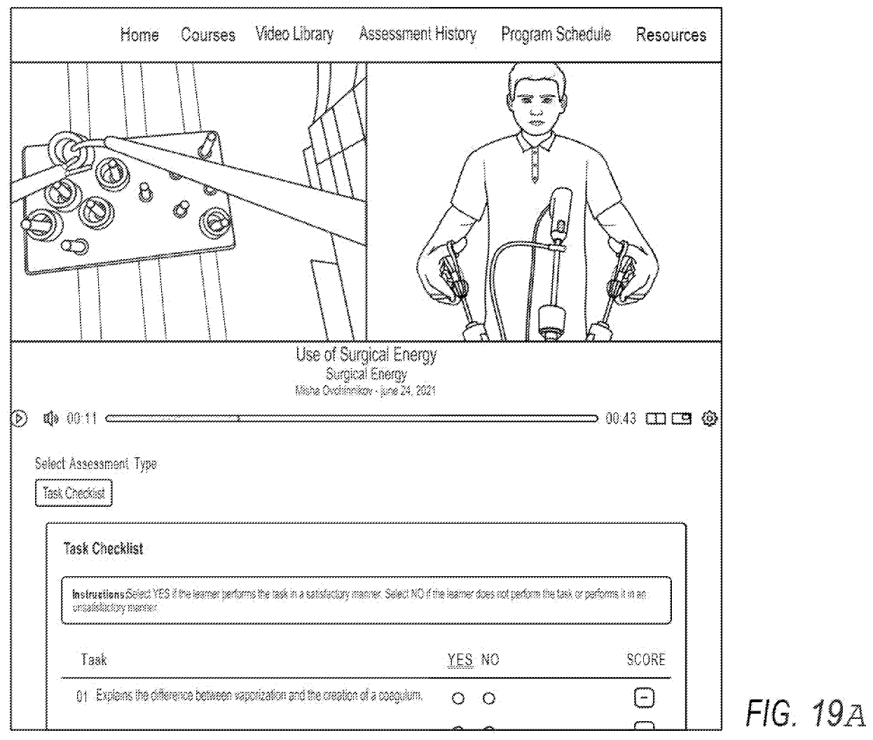

In various embodiments, the video assessment module 440 can be configured to selectively connect to one or more of the internal or external A/V sources 130, 140 associated with a surgical trainer 110. An exemplary view illustrating user selection and connection of one or both internal/external A/V sources can be seen in FIG. 18.

In various embodiments, the video assessment module 440 is configured to capture and/or record the data simultaneously from the internal input source 130 (e.g., audio and/or video within or inside the surgical trainer 110) with data from the external input source 140 (e.g., audio and/or video directly outside and proximal to an outer surface of the surgical trainer 110). As such, via a multi-feed (e.g., dual-feed) capturing and/or upload process provided by the video assessment module 440, users (e.g., trainees, residents) are able to simultaneously capture and/or record their surgical performance directly from multiple audio and/or video sources (e.g., internal and external A/V sources 130, 140). Exemplary internal A/V sources 130 may include a first camera or laparoscope. Exemplary external A/V sources 140 may include a second camera or webcam. In various embodiments, both the internal and external A/V sources 130, 140 are directly connected to the surgical trainer 110 or a client processor 160 facilitating communication between the internal/external input sources 130, 140, and the surgical trainer 110, and/or the surgical simulation system 120. In various embodiments, the video assessment module 440 is configured to provide one or more views or tools with pre-populated and/or user populated or selectable data providing the viewing, capturing and/or submission of a user's surgical performance. The information being captured by the selected internal and/or external A/V sources can be shown on the display 250 of the surgical trainer 110 or on associated computing devices. In various embodiments, the video assessment module 440 may be instructed (via user selection and/or automatically performed) to directly connect to one or more surgical simulation system 120 and transfer the captured data directly (i.e. stream) to the surgical simulation system 120.

In various embodiments, the video assessment module 440 stores the separate multiple audio/video data separately but may be configured to provide the separate multiple audio/videos in a synchronized form upon playback by the user and/or for assessment. In various embodiments, the video assessment module 440 may be configured to combine or stitch two separate audios/videos into a single-stitched audio/video file. The single-stitched audio video file ensures that the two separate audios/videos remain synchronized ensuring that surgical operations or manipulations performed by the user inside the surgical trainer matches are at least are in-sync with the corresponding surgical operations or manipulations performed by the user on the outside of the surgical trainer 110. In various embodiments, the single-stitched audio/video file further simplifies handling, manipulation, or storing of the audio/video associated with the recording of the surgical simulation performance performed by the user.

In various embodiments, the video assessment module 440 is configured to save, store, and/or submit a capture of the data associated with the user's surgical performance (e.g., one or more audio/videos, a single-stitched performance audio/video, or separate synchronized audio/videos) for assessment or evaluation by one or more assessors. In various embodiments, the video assessment module 440 can be configured to provide an upload or storage process and/or instructions to the user to upload and/or store the user's surgical performance with the surgical simulation system 120 and/or the surgical simulation storage 170 which were obtained from alternative sources different from the internal and/or external A/V sources 130, 140.

In various embodiments, the video assessment module 440 assesses and/or is capable of carrying out the procedure associated with an assessment of a user-submitted A/V capture of data associated with the surgical performances with or without user interaction. In various embodiments, the video assessment module 440 is configured to provide some form of notification (e.g., via an email or text message) to an assessor and/or the user, that a captured surgical performance (e.g., multimedia data) has been submitted by a user and available for review and/or an assessment of such a submitted surgical performance has been completed by an assessor/feedback is available. The notification process can also be communicated from the surgical simulation system 120 (e.g., coordinated by the video assessment module 440 and/or the main control processor 400) and provided to the respective one or more surgical trainers 110, client processors 160, and/or assessor systems 150.

In various embodiments, the video assessment module 440 can also be configured to provide access to a user's surgical performance to be assessed by the user as well as others (e.g., peers, faculty, and/or experts). In various embodiments, the video assessment module 440 is configured to prevent the user from reviewing their own user performance data. Rather, the video assessment module 440 can be configured to notify random a pre-determined number of assessors from a pool of eligible assessors. In another embodiment, the assessors can be notified from a pre-determined list of assessors. The notification is performed once the user performance data has been uploaded to the surgical simulation system 120. In various embodiments, the user is allowed to do a self-evaluation and such information associated with the self-evaluation can be labeled accordingly so as to distinguish itself from the information (e.g., feedback) provided by assessors.

In various embodiments, the video assessment module 440 can be configured to select a pre-defined assessment tool with or without user interaction (e.g., automatically). The assessment tools are designed to illustrate/describe to the assessors how a particular surgical simulation should be performed and thus allow the assessors some points on how to assess any one user's performance from their performance data. The selected pre-defined assessment tool is used in conjunction with the assessment of the captured surgical performance video. The video assessment module 440 in various embodiments is configured to determine and select the appropriate pre-defined assessment tool based on course data, activity data, and the like. In various embodiments, for example, criteria and likewise the assessment tool or form for assessing one user (e.g., a resident) may be different compared to a different user (e.g., a practicing doctor). Thus, the video assessment module 440 can be configured to identify situations where differences in the criteria being used to assess user performance data may be introduced, for example based on an amount of experience that the user may have with the particular surgical simulation being performed.

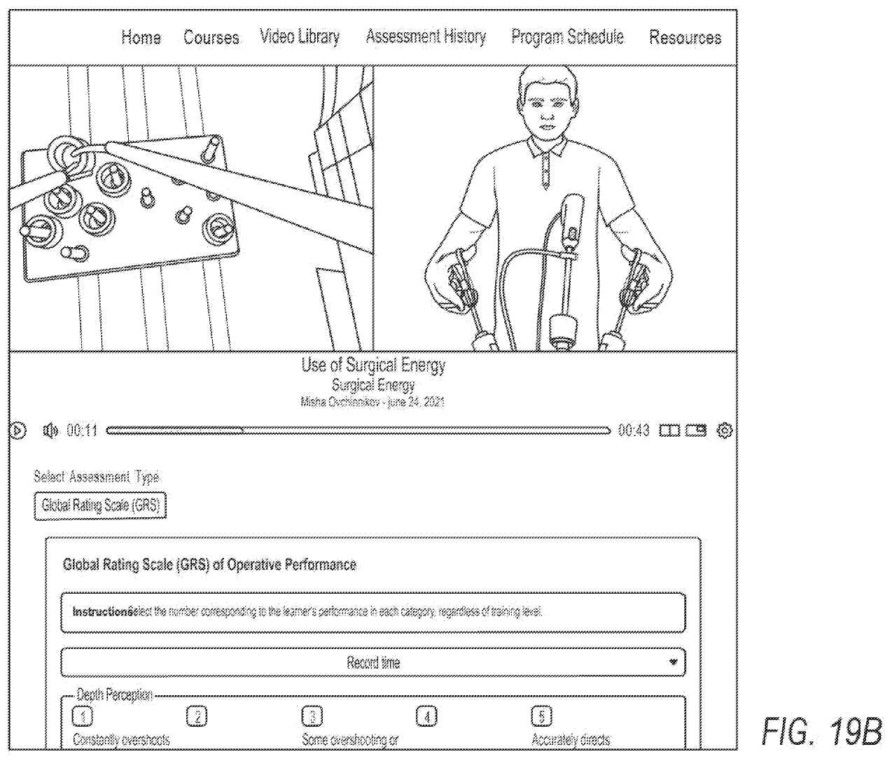

In various embodiments, the video assessment module 440 is configured to provide the performance video simultaneously with the assessment tool thereby allowing for simultaneous (i.e. real-time) interaction of the performance video with the assessment tool. As such, assessors (such as faculty or peers) would be able to initiate a playback a recording of the user's surgical performance via the video assessment module 440 as synchronized videos or a single-stitched video file generated by the video assessment module 440 while simultaneously assessing them using faculty assessment data. In some embodiments, the faculty assessment data may include but would not be limited to data from Accreditation Council for Graduate Medical Education (ACGME)-approved assessment forms like the Global Operative Assessment of Laparoscopic Skills (GOALS) assessment, selected and/or provided by the video assessment module 440 with or without user interaction. In various embodiments, the video assessment module 440 provides one or more views or tools with selectable pre-populated and/or user populated data providing the viewing and assessment of a user's surgical performance and/or submission of such assessments. Such exemplary views of a real-time video assessment being performed on user surgical performance data is shown in FIGS. 19A-B.

In the example view of FIG. 19A and FIG. 19B, an assessor would be able to review the audio and the video simultaneously on the top of user interface. The assessors may be able to fast forward, reverse, skip, and/or pause the video/audio. Other features provided in the view may allow the orientation of the video to be aligned on one side (e.g., left/right half) of the screen as opposed to top half or even minimized to a corner.

On the bottom of the user interface would include various questions which would guide a user what to look for in the user performance data in order to assess the user's performance/competency. The questions may form a list of tasks that the user may need to perform during the surgical simulation and ask the assessor to quantify how well the task was performed. In other embodiments, the assessor may be asked to quantify the user's skill performance on a scale (e.g., 1-5) with the scale including some guidelines on what quantifies performance at a particular level.

Although the figures illustrate pre-populated guidelines and inputs that the assessors can use to assess the user's performance from the user performance data, other embodiments may allow the assessors more freedom on characterizing and explaining the characterization of the user's perceived competence from the user performance data. For example, an assessor may be capable of providing their own comments on various shortcomings seen in the video as well as suggestions on how to better perform a particular task.

In various embodiments, assessors may be capable of selecting the type of feedback they would like to provide (e.g., pre-populated or user populated). An option may allow the assessors the choice. In various embodiments, assessors may be instructed to provide input that consists of both pre-populated and user populated inputs.

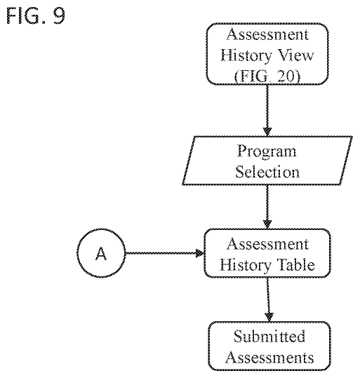

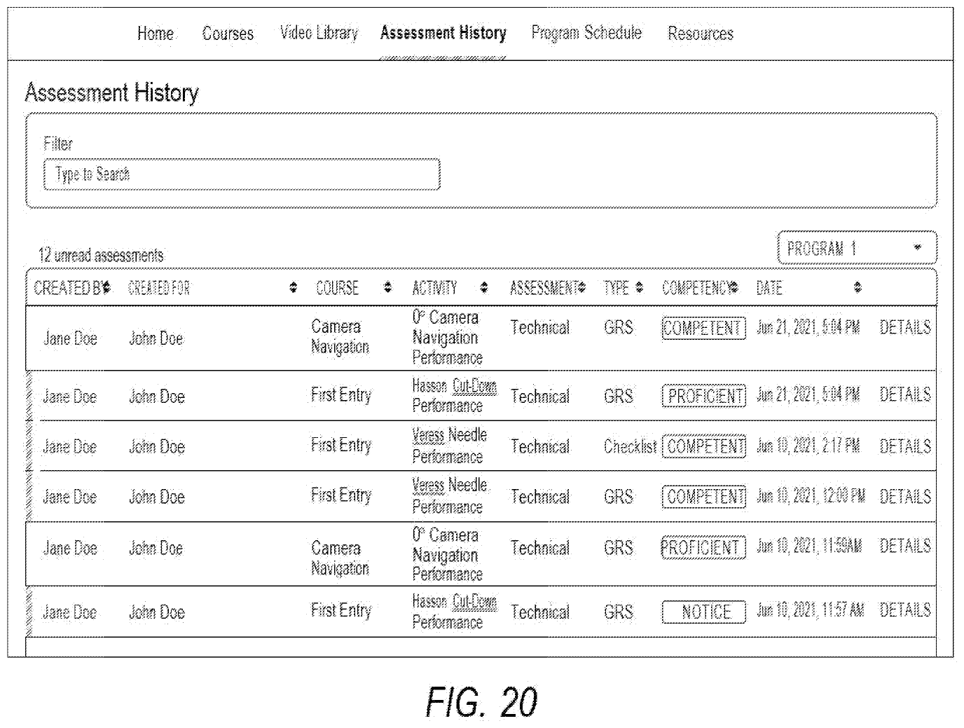

In various embodiments, the assessment history module 450 comprises the compiled assessment data generated by the assessment module 435 of the course module 430 and/or the video assessment module 440. Such data can be stored in the surgical simulation controls/data storage 170 and be accessible directly by the assessment history module 450 and/or through the main control processor 400. In other embodiments, the complied assessment data can be stored within the surgical simulation system 120. Exemplary operations performed by the assessment history module 450 are provided in FIG. 9. Furthermore, an exemplary view of the assessment history provided by the assessment history module 450 can be seen in FIG. 20. The exemplary view provides a list of uploaded user performances related to various surgical simulations performed by the user. Furthermore, the list may also include information such as the type of surgical simulation, who created the surgical simulation, and the feedback (e.g., competency) of the user. Additional details can also be accessed via a further link or button from this view for one or more of the uploaded user performances displayed in the list.

The surgical simulation system 120 (e.g., via assessment module 435 via the course module 430, the video assessment module 440, and/or the assessment history module 450) are configured to eliminate the need for an assessor, such as a faculty member, to be present or be in the same physical location as the trainee (i.e. the user of the surgical trainer performing the surgical simulation) in order to provide an assessment of the user's surgical performance of a predetermined or pre-selected procedural or skill-based exercise. The surgical simulation system 120 provides a centralized system to capture, process, store, and access video and/or audio data (e.g., multimedia, recorded surgical performances). As such, the surgical simulation system 120 can be used to address the issue of faculty members' limited time to schedule in-person meetings with residents and likewise increase the opportunity for residents to receive more performance assessments, instruction, and training as well as providing such assessment, instructions, and training with less delays (e.g., time between submission of captured data of a surgical simulation performance and receiving feedback from one or more assessors). The use of the surgical simulation system 120 is not limited to just training students. In various embodiments, hospitals can also utilize the system to help update and train newer medical professionals (e.g., surgeons) while having other more experienced medical professionals assess and provide feedback accordingly.

The surgical simulation system 120 is configured to receive, store, and organize user surgical performance data and associated peer assessments of each performance data. The surgical simulation system 120 is also configured to handle the accessibility and distribution of the user surgical performance data between potentially other surgical simulation system 120, the one or more surgical trainers 110, the one or more client processors 160, and/or the one or more assessor systems 150 all within the same overall surgical simulation network system 100. In this way, the surgical simulation network system 100 is configured to provide a single surgical simulation training system and thereby avoids the complications provided by, for example, third-party systems, storage, or applications.

As described above, embodiments for the surgical simulation network system 100 are configured to facilitate remote assessors to review and provide feedback to a user's surgical simulation performance which have been captured and uploaded to the surgical simulation network system 100. This generally covers the streamlining of the evaluation process by allow for the assessors to remotely access, review, provide feedback, and forwarding that feedback from the assessor to the respective user for the uploaded user surgical performance data. This feature provides assessors more freedom (e.g., less time-constraints) regarding when the assessors can review and provide feedback to the users regarding surgical simulations since there is no longer a limitation regarding the assessor needing to be present or even at the same physical location (e.g., school, hospital) as the user to review and evaluate user performance. Thus, for example, a doctor at a hospital in one state can evaluate and provide feedback for a resident at a different hospital in a different state.

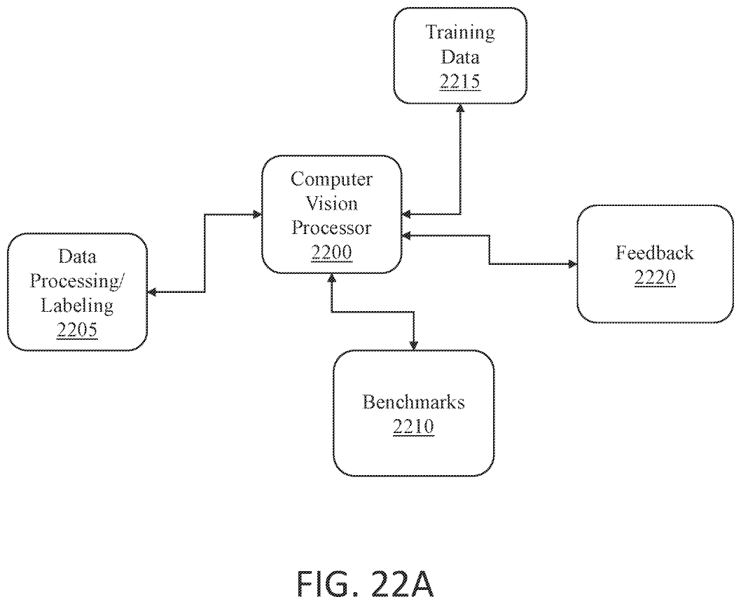

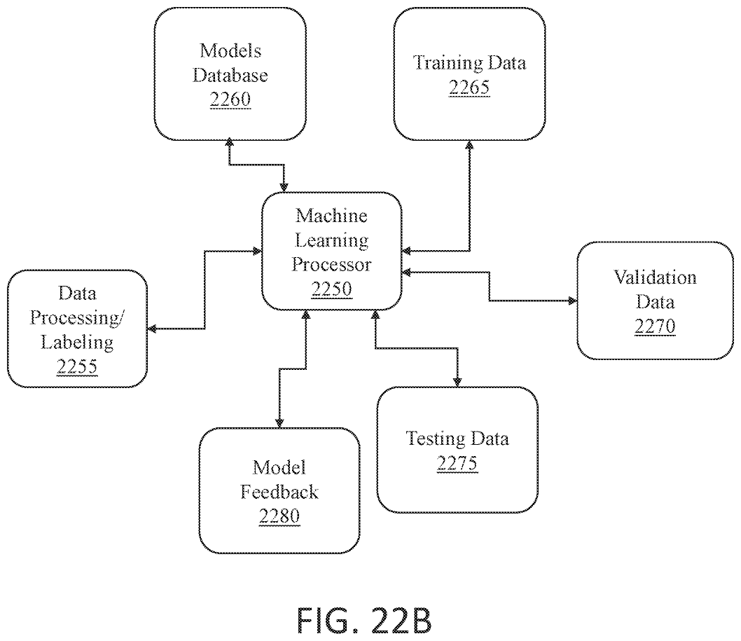

In various embodiments, the surgical simulation network system 100 of FIG. 1 may further comprise one or both of a computer vision module (as illustrated in FIG. 22A) or a machine learning module (as illustrated in FIG. 22B) to further automate the review, assessment and/or evaluation of user surgical simulation performance data. In some embodiments, the automated functionality provided via the computer vision and machine learning for the surgical simulation system 100 can be used in conjunction with the review, assessment and/or evaluation provided by assessors (e.g., faculty). In various embodiments, the surgical simulation network system 100 can be configured to be fully automated without the need for or interaction by assessors to perform any review, assessment and/or evaluation of user surgical simulation performances. The automated nature, either fully or partially provides feedback more rapidly back to the user since the wait time for an assessor to perform the review, assessment and/or evaluation of the surgical performance is reduced or removed. Furthermore, the review, assessment and/or evaluation of the user's performance can be more objective and more consistent across many different users, as, for example, the same computer vision and/or machine learning modules using the same models/criteria to assess each user's performance can be used.

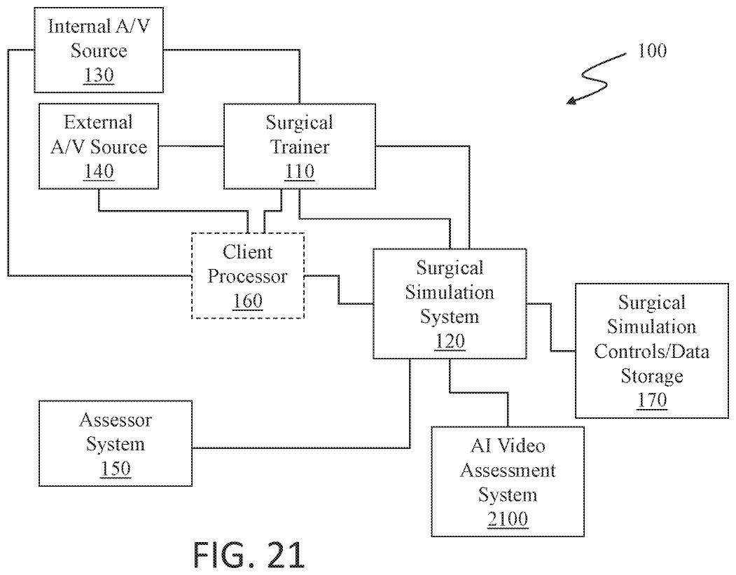

FIG. 21 illustrates an embodiment where an artificial intelligence (AI) video assessment system 2100 is provided with the surgical simulation network system of FIG. 1. As illustrated in the figure, the AI video assessment system 2100 may be a separate system that communicates with the surgical simulation system 120 in order to provide the surgical simulation network system 100 with the capability to automate various processes/operations such as processing surgical simulation performances, identifying what surgical simulation is being performed by the user from the surgical simulation performance, identifying and labeling various points of interest within the surgical simulations and/or performances, providing feedback by comparing the various points of interest within the surgical simulations and/or performances with pre-determined benchmarks, and subsequently generating training data that can be used with machine learning. In some embodiments, the AI video assessment system 2100 may be, partly or in its entirety, implemented in one or more of the assessor systems 150 and/or the surgical simulation system 120. Further details regarding the computer vision module and the machine learning module associated with the AI video assessment system 2100 will be provided below in connection with FIG. 22A and FIG. 22B.

With reference to FIG. 22A, the figure illustrates the aspects of the computer vision module associated with the AI video assessment system 2100. The computer vision module comprises a computer vision processor 2200, which is connected to or integrated with one or more separate modules, sub-systems, and/or sub-processors. Exemplary modules, subsystems, and/or sub-processors include (but are not limited to) a data processing/labeling module 2205, benchmarks database 2210, training data module 2215, and/or a feedback module 2220.

The computer vision processor 2200 coordinates the operation and communication between the different modules, sub-systems, and/or sub-processors of the computer vision module to implement computer vision functionality for the surgical simulation network system 100. In various embodiments, the computer vision processor 2200 can be configured to retrieve from and/or store operational, control, and/or informational data into the surgical simulation controls/data storage 170.

The data processing/labeling module 2205 uses the surgical simulation performance from the surgical simulation network system 100 provided by its various users. The uploaded surgical simulation performance are captured using various A/V sources (e.g., external, internal) to capture user performance data of a user performing a surgical simulation. The data processing/labeling module 2205 is configured to process the captured data that was captured and subsequently uploaded to the surgical simulation network system 100 and labels various points of interest within the captured data as processing is performed. The processing performed by the data processing/labeling module 2205 may include the use of a variety of filters to, for example, modify images in the surgical simulation performances so that various objects can be identified. Such processing can be performed, for example, on a frame by frame and pixel by pixel basis. This allows the data processing/labeling module 2205 to identify objects within the data being processed which can be useful in identifying the type of surgical simulation being performed (e.g., based on the position of the obstacles/interactable elements) as well as identifying user movement to characterize the user's performance (e.g., movement of the user manipulable instrument such as a grasper).

The data processing/labeling module 2205 can implement (e.g., embed) one or more pre-defined labels within the processed surgical simulation performance data which can be processed, outputted, and used by the computer vision processor 2200. For example, the computer vision processor 2200 can be configured to track the location of a particular object or the user's maneuvers during the surgical simulation with the use of the labels. The labels assigned by the data processing/labeling module 2205 can also be referenced with data stored in the benchmarks database 2210 that could be useful in providing an evaluation of the user's performance/competency.

The processing performed by the data processing/labeling module 2205 can also include any number of pre-processing necessary for the captured surgical simulation performance to be usable with the AI video assessment system 2100 (or generally with the surgical simulation network system 100). The pre-processing for the data processing/labeling module 2205 may include data cleaning/cleansing, data integration, data transformation, and data reduction. For example, pre-processing can be performed to transform captured surgical simulation performances into a pre-determined format being used by the surgical simulation network system 100. This may come up, for example, because captured surgical simulation performances may be obtained from different surgical trainers each with different types of A/V sources, which in turn may have different A/V formats. Having a standardized format for all the captured surgical simulation performances being subsequently processed by the surgical simulation network system 100 can simplify compatibility concerns associated with the subsequent review, assessment and/or evaluation of the surgical simulation performances performed at different user devices by the assessors.

The benchmarks database 2210 contains data that is used (via the feedback module 2220) to assess and evaluate the surgical simulation performance. For example, the benchmarks database 2210 can include various pre-defined thresholds and/or models that can be used by the computer vision processor 2200. Some of these types of benchmarks are used to compare points of interest found in the surgical simulation performance in order to positively identify specific points of interest in the data (e.g., identifying particular objects). The identified objects can be used to identify a particular surgical simulation being performed by the user (e.g., arrangement of objects that users can interact with) without the need for the user to provide such identifying information to the surgical simulation network system 100. The benchmark database 2210 can also include models that can be used to identify user manipulable objects (e.g., laparoscopes, graspers) which users would utilize during the performance of the surgical simulation. Via the use of the latter types of benchmarks, the computer vision module can then track the user's movements within the surgical simulation performance. Further thresholds and/or models included in the benchmark database 2210 can also identify preferred motions/movements that the user should be taking during a particular surgical simulation. These thresholds and/or models allow for the computer vision module to assist in the automated evaluation of the user performance via the feedback module 2220.

In some embodiments, other types of benchmark data can evaluate other factors or metrics such as, but not limited to, identifying how fast the user takes to complete the surgical simulation, smoothness of motion, economy of motion, and force. These other types of benchmark data can further facilitate in the evaluation of the user's performance within the surgical simulation data being evaluated. Exemplary details regarding how some of these factors or metrics can be provided are described in U.S. patent application Ser. No. 15/895,707 (entitled “Laparoscopic Training System”) which is incorporated herein by reference in its entirety.

In some embodiments, there may be two or more different sub-set of thresholds and/or models which have different criteria. The different criteria may correspond to different difficulties thus coinciding with different groups of users. For example, a more lenient/easier set of thresholds and/or models may be assigned to more novice users (e.g., residents) whereas more harsh/harder set of thresholds and/or models may be assigned to more experienced users (e.g., practicing doctors).

The computer vision module can also be configured to generate training data via the training data module 2215 based on the processing of each user's surgical simulation performance data. The training data from the training data module 2215 can then be used by the machine learning module (as illustrated in FIG. 22B) to train/update one or more models used by the AI video assessment system 2100. In connection with the validation and testing data sets (discussed below), the AI video assessment system 2100 can maintain up-to-date models that are usable to automatically review, assess, and/or evaluate surgical simulation performances on the surgical simulation network system 100.

The feedback module 2220 is configured to assess user performance from the surgical simulation performance data and comparing the surgical simulation performance data against the data stored in the benchmarks database 2210. As discussed above, the feedback module 2220 can utilize labels implemented/embedded into the surgical simulation performance data provided by the data processing/labeling module 2205 to process, review, and assess the performance data in conjunction with the benchmarks database 2210. Based on the comparisons, the feedback module 2220 is configured to provide various outputs (e.g., feedback) to the user. In one embodiment, the feedback module 2220 provides an overview of the user's performance (e.g., competency) in connection with the surgical simulation performance data that was reviewed. In other embodiments, the feedback module 2220 can provide suggestions on how to perform the surgical simulation better. The feedback provided by the feedback module 2220 can be provided in a variety of different ways such as via email or displayed on a screen of the surgical trainer 110, the client processor 160 and/or the assessor system 150. The overview provided by the feedback module 2220 can include detailed breakdown of one or more factors that may have been considered by the AI video assessment system 2100 when assessing/evaluating the user's performance in the surgical simulation performance data. For example, a user's time of completion can be shown compared with the “ideal” time of completion. Other comparisons can also be provided which show the user's performance in comparison with the corresponding “ideal” benchmark (e.g., path). In some embodiments, only the user's overview data may be shown with no reference to the “ideal” benchmark. The user's overview data can also be displayed in comparison with data of one or more other users. For example, the user's overview data may be used to show that the user has performed a particular section or skill better than a percentage of previous assessed users (e.g., faster than x % of other users).

In some embodiments, the surgical simulation network system may be capable of capturing and transmitting the user's surgical simulation performances from the surgical trainers to the surgical simulation system in real-time (i.e. streaming) or close to real time. In doing so, the AI video assessment system 2100 may be configured to process the surgical simulation performance data and provide real-time or close to real-time feedback related to the user's performance. For example, based on the user's positioning of the user manipulable device, real-time feedback can be provided such as a light on the device providing a colored response based on the satisfactory (e.g., green color) or unsatisfactory (e.g., red color) positioning of the user based on the assessment being made by the AI video assessment system 2100. In one embodiment, the color can be used to ensure that the user is moving the device at an appropriate speed. In another embodiment, the color can be used to ensure that the user is moving the device in an appropriate path. In another embodiment, the color can be used to identify if the device comes into contact with obstacles (e.g., tissue) in order to enforce manual control of the device of where the user can move the device. In another embodiment, the real-time feedback may be capable of providing feedback in real time that is superimposed over the captured data being displayed on a separate display screen. In this way, the user (and other viewers) can view how the AI video assessment system is evaluating the user's performance in real time. Other types of feedback (e.g., audio, haptic) are also possible in various embodiments to convey the status of the user's performance in real-time or near real-time. For example, there may be speakers or motors on the device that exhibit a sound or cause a vibrating response in response to pre-determined conditions being detected.

FIG. 22B illustrates further details regarding the machine learning module associated with the AI video assessment system 2100. The machine learning module provides the surgical simulation network system 100 the capability of being able to update and improve the various benchmarks (e.g., models, thresholds) used to identify the surgical simulations being performed by the user and used to assess/evaluate the surgical simulation performance data for feedback purposes. The improvement achieved via machine learning for surgical simulation network system 100 occurs over time as the machine learning module utilizes all the available incoming data (e.g., the surgical simulation performance data by the various users) to continually update the benchmarks stored, for example, in the benchmarks database 2210. In some embodiments, the benchmarks used to review and evaluate users on various surgical simulations can be updated internally within the surgical simulation network system 100 without the need for external assistance.

With reference to FIG. 22B, the machine learning module comprises a machine learning processor 2250, which is connected to or integrated with one or more separate modules, sub-systems, and/or sub-processors. Exemplary modules, sub-systems, and/or sub-processors include (but are not limited to) a data processing/labeling module 2255, models database 2260, training data module 2265, validation data module 2270, testing data module 2275, and a model feedback module 2280.

The machine learning processor 2250 coordinates the operation and communication between the different modules, sub-systems, and/or sub-processors of the machine learning module to implement machine learning functionality for the surgical simulation network system 100. In various embodiments, the machine learning processor 2250 can be configured to retrieve from and/or store operational, control, and/or informational data into the surgical simulation controls/data storage 170. The same data can also be stored with the surgical simulation system 120 in which the machine learning processor 2250 can also be configured to locate and retrieve.

The data processing/labeling module 2255 uses the surgical simulation performance data from the surgical simulation network system 100 similar to the data processing/labeling module 2205 associated with the computer vision module. For example, the data processing module 2255 is also configured to perform any necessary pre-processing of the data to provide uniformity. This reduces the amount of subsequent post-processing needed in connection with the creation and maintenance of the machine learned models stored in the models database 2260.

The post-processing performed by the data processing/labeling module 255 similarly also processes the surgical simulation performance data to identify various points of interest and apply one or more pre-determined labels. In some embodiments, the processing and labeling of the surgical simulation performance data can be performed with respect to a single frame, a range of frames, and/or via pixels.

With the labels in place, in various embodiments, the training data module 2265 can use the processed surgical simulation performance data to generate a training data set. The training data set will be used to update an existing model stored in the models database 2260 or generate a new model if one such model does not exist in the models database 2260.

When a new model has been created or an existing model is updated, the validation data module 2270 performs a validation step to verify whether the newly generate/recently updated model is satisfactory (i.e., operates as intended). This is performed via use of one or more validation data set(s) that is associated with the validation data module 2270. If the output of the newly generated/recently updated model is satisfactory with the use of the validation data set, then the machine learning module performs one last test. However, if the output of the newly generated/recently updated model is not satisfactory, the newly generated/recently updated model is discarded and the machine learning module may, in some embodiments, start the machine learning process from the beginning with the next data set(s) and the models stored in the models database 2260. What is deemed satisfactory can be pre-determined or at some point established, for example, by the administrator of the surgical simulation network system 100.

Once the newly generated/recently updated model has been sufficiently validated, the newly generated/recently updated model can undergo one last verification via the testing data module 2275. The testing data module 2275 contains a testing data set that will be used to confirm the satisfactory operation of the model. If the model's output after the use of the testing data is acceptable, the newly generated/recently updated model is stored in the models database 2260 so it can be used for future assessments. However, if the testing data provides an invalid output, the newly generated/recently updated model is be discarded and the machine learning process performed by the machine learning module begins again with the existing model(s) in the models database 2260 and the next data set(s) (e.g., user uploaded surgical simulation performance data).

As discussed above, the newly generated/recently updated models stored in the models database 2260 can be used to better identify points of interest in the user surgical simulation performance data as well as improve the automated assessment of the user's performance. In various embodiments, assessors are able to provide their assessments alongside the assessments provided by the AI video assessment module 2100.

The model feedback module 2280 is configured to assess user performance from the surgical simulation performance data using one or more models stored in the models database 2260. The model feedback module 2280 can utilize labels implemented/embedded into the surgical simulation performance data provided by the data processing/labeling module 2255 in order to review and assess the surgical simulation performance data in conjunction with the models stored in the models database 2260. Based on the comparisons, the model feedback module 2280 is configured to provide various outputs (e.g., feedback) to the user. In one embodiment, the model feedback module 2280 provides an overview of the user's performance (e.g., competency) in connection with the surgical simulation performance data that was reviewed/assessed. The feedback provided by the model feedback module 2280 can be provided in a variety of different ways such as via email or displayed on a screen of the surgical trainer 110, the client processor 160, and/or the assessor system 150. The overview provided by the model feedback module 2280 can include detailed breakdown of one or more factors that may have been considered when assessing/evaluating the user's performance in the surgical simulation performance data. For example, the model feedback module 2280 can be configured to display a comparison between the user's performance with the model used for the feedback. In some embodiments, only the user's feedback-related data may be shown with no reference to the model being used. In some embodiments, the user's feedback-related data can also be displayed in comparison with feedback-related data of one or more other users. For example, the user's feedback-related data may be used to show that the user has performed a particular section or skill better than a percentage of previous assessed users (e.g., faster than x % of other users).

FIG. 23 illustrates exemplary operations performed by the video validation module 460 of the surgical simulation system 120. As described above, in connection with the surgical simulation system 120 as illustrated in FIG. 4, various embodiments may have the user performance data streamed directly from one or more surgical trainers 110 to the surgical simulation system 120. In various embodiments, the surgical simulation system 120 is associated with one or more computing systems located at a physical location. However, other embodiments may have one or more surgical simulation systems 120 operate entirely through the Internet (i.e. be cloud-based) which takes advantage of available computing processing resources and data storage capabilities provided by data centers located at many locations around the world.

With the streaming of the data from the surgical trainers to the surgical simulation system 120, the video validation module 460 provides a means by which the surgical simulations system 120 can evaluate whether the user performance data being received (i.e. streamed) from one or more surgical trainers 110 is satisfactory/appropriate. This enables the user performance data to be in a state such that it can be properly processed, stored, and subsequently recalled for assessment. If one or more issues are detected (i.e. missing packets, different formats), the issues can be addressed by the video validation module 460 (e.g., via conversion or repair) in order to attempt to place the user performance data in a condition that is satisfactory. Otherwise, the video validation module 460 is configured to request resubmission of the data (e.g., another data set, repeat of the previously submitted data set) from the user associated with the user performance data at issue to acquire a data set that is satisfactory/appropriate.

Returning to FIG. 23, the figure illustrates an embodiment of the video validation module 460 having various steps/operations that are performed on user performance data that are recorded/captured and subsequently submitted by the surgical trainers 110 to the surgical simulation system 120. It should be noted that in other embodiments, there may be more steps/operations, less steps/operations, and/or the steps/operations may be performed in a different order that what is currently illustrated in FIG. 23. However, the overall purpose of the video validation module 460 is to ensure that the user performance data that will be ultimately stored on the surgical simulation network system 100 is in a state that is compatible with the surgical simulation network system 100 (e.g, processed, stored, and recalled for assessment).

Once the surgical trainer begins capturing user performance data and transmitting/stream the data to the surgical simulation system, an initial evaluation of the user performance data is performed by the video validation module 460 (via step 2310). In various embodiments, similar to what has been discussed above, the video validation module 460 is associated with the surgical simulation system 120 used to evaluate the user performance data as it is being transmitted or streamed directly from the surgical trainers 110 (i.e. captured via internal and/or external A/V sources) to the surgical simulation system 120. However, other embodiments may have similar video validation features associated with the surgical trainer 110 (or client processors 160) that would be used to evaluate whether the data being captured and stored at the surgical trainer 110 is satisfactory/appropriate prior to transmission to the surgical simulation network system 100. Having the video validation features at the surgical trainers 110 can be provided for embodiments where the user performance data is first captured and stored locally at the surgical trainers 110 before being transmitted to the surgical simulation system 120 at a later time. An advantage is that this may allow various processes (e.g., formatting, conversion, repairs) to be performed locally instead of all in the surgical simulation network system 100.

Once the user performance data has been received by the surgical simulation system 120, the video validation module 460 can evaluate (in step 2310) the format of the user performance data. For example, the video validation module 460 can evaluate whether the user performance data is being captured in a pre-determined format (e.g., H264). If not, the video validation module 460 may take steps to process the user performance data (e.g., VP8) so that the user performance data can be placed in a compatible format that can subsequently used by the simulation network system 100.

After the format of the user performance data has been evaluated in step 2310, the user performance data as a whole can then be subsequently provided/uploaded to the surgical simulation network system 100 in step 2320 for further processing. Once the user performance data is being uploaded to the surgical simulation system 120, the data can further be evaluated to determine its integrity (in step 2330) by the video validation module 460. For example, the evaluation of the user performance data integrity comprises determining whether the user performance data is complete (i.e. no missing portions of data). In scenarios where the communication (i.e. wireless Internet connection) between the surgical trainers 110 and the surgical simulation network system 100 are unreliable, portions/packets of the user performance data may be out of order or missing (i.e. dropped). The upload step 2320 performs numerous checks to determine whether all the data packets associated with the user performance data are present and in the appropriate order. When the video validation module 460 determines that the user performance data is no longer being transmitted (i.e. the user has finished recording), if there are missing portions of the user performance data, the video validation module 460 can request those portions (which may be stored locally in a cache simultaneously during transmission to the surgical simulation system 120) to be resubmitted from the surgical trainer 110. In addition, the portions of the user performance data that is out of order can also be identified by the video validation module 460. In both cases if issues have been identified with the user performance data, the video validation module 460 is configured to perform repairs in step 2340. For example, the video validation module 460 is configured to reconstruct the video file into the sequence that corresponds to what was captured by the internal and/or external A/V sources at the surgical trainer 120. This may include reordering the packets of data and/or inserting the missing packets into the sequence for the user performance data.

The video validation module 460 can also be configured to check other aspects of the user performance data being provided by the surgical trainers 110 to validate that the user performance data is satisfactory/appropriate in step 2330. For example, the video validation module 460 may determine whether the user performance data is of an appropriate size. It may be a concern to ensure that the user performance data is below a pre-determined size. If the data being submitted is too large, the video validation module 460 can be instructed to request the user to submit a different data set that satisfies the data size restriction.

Once the integrity of the user performance data has been validated or repaired, the user performance data can then be subsequently stored in the surgical simulation network system 100 (in step 2350). The storage of the user performance data allows subsequent retrieval of the same data for assessment by one or more assessors via the assessor system 150. In various embodiments, the video validation module 460 is configured to store the user performance data into the data storage 170 of the surgical simulation network system 100.

In various embodiments, the video validation module 460 is also configured to standardize the format of the user performance data that has been stored on the surgical simulation network system 100. For example, the video validation module 460 can standardize the user performance data format so that the user performance data can be displayed on different computing devices (e.g., laptops, mobile devices, personal computers). In various embodiments, the video validation module 460 can standardize the user performance data format so that the user performance data can be displayed on one or more browsers. The standardization of the format for the user performance data allows control over where the user performance data can be viewed (e.g., via specific browsers or computing devices) for subsequent assessment.

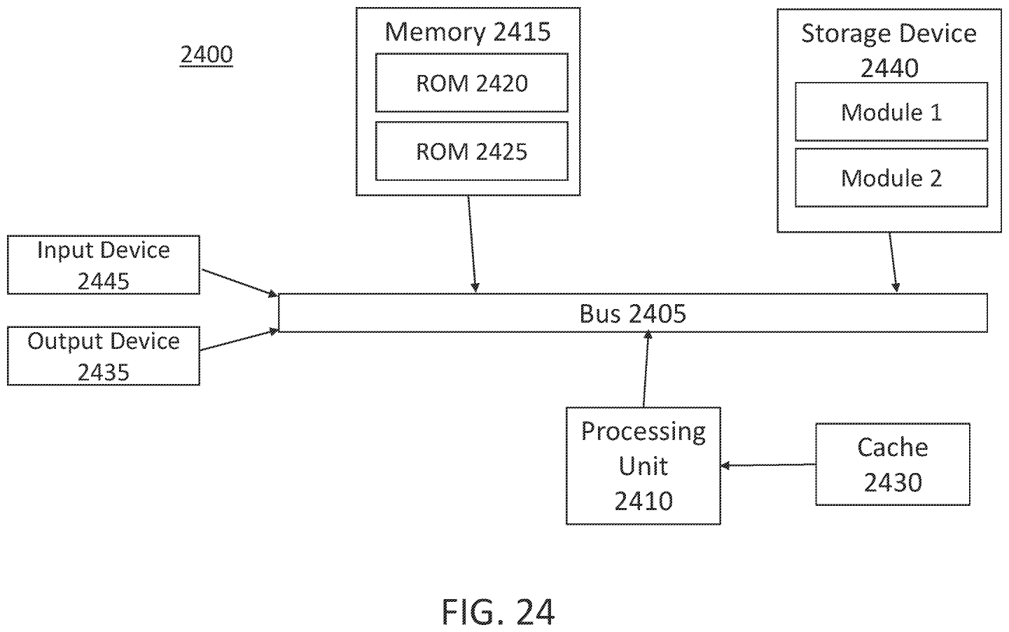

With reference to each of its components, the many described systems and processors of the present disclosure, FIG. 24 illustrates an example computing system 2400 used in connection with such systems and processors to carry out the surgical simulation network system 100 described within this application. For example, the example computing system 2400 illustrated in FIG. 24 may correspond to a computing system used in the implementation of one or more of the surgical trainers 110, surgical simulation systems 120, assessor systems 150, AI video assessment systems 2100, and/or the client processors 160. Although the following description provides details related to a particular embodiment of a computing system used for the surgical simulation network system comprising a particular set of components arranged in a particular manner, other embodiments are possible and one of ordinary skill in the art will understand that the other embodiments are possible which still maintains the intended functionality of the surgical simulation network system.

Referring back to FIG. 24, the figure illustrates an example architecture for the computing system 2400 having a number of different components that are in communication with each other. However, other embodiments may have less, more, or different components that are configured to facilitate a desired operation of capturing surgical simulation performance data and assessing user performances based on processing and reviewing the captured data.

First, the computing system 2400 can include a processing unit (CPU or processor) 2410. The processing unit 2410 is configured to process, for example, incoming data (e.g., input devices 2445), execute and perform calculations via instructions stored in memory 2415, and provide an appropriate output (e.g., to the output device 2435). The processing unit 2410 can also be configured to manage how the various components (e.g., input device 2445, output device 2435) associated with the computing system 2400 operate.

A system bus 2405 may also be included to provide communicative coupling between the various components associated with the computing system 2400 with one another. In particular, the system bus 2405 provides a means by which data can be transmitted between each of the components in order to allow the computing system 2400 to carry out its functions/operation. For example, the system bus 2405 may couple other components such as memory 2415 to the processing unit 2410 which allow for the transfer of data and instructions between the memory 2415 and the processing unit 2410. The system bus 2405 can also be used to transmit the user input obtained from the input device 2445 to the memory 2415 so the user input can be stored or to the processing unit 2410 so that the user input can be processed. Subsequently data from the processing unit 2410 (i.e. feedback) can be transmitted via the system bus 2405 to the output device 2435 so that, for example, users can view the information produced by the computing system 2400 (or in some cases the surgical simulation network system 100 as a whole). Such information may include, for example, the state of a surgical simulation being performed, the status of the performance data that was captured and transmitted to the surgical simulation network system 100 and/or the user's competency in performing the surgical simulation.

The memory 2415 of the computing system 2400 can be used for storage of a variety of different data sets. The memory 2415 may include a combination of read only memory (ROM) 2420 and/or random-access memory (RAM) 2425. In some embodiments, the memory 2415 may also include flash memory.

The memory 2415 can store various modules that house modules (e.g., programs made of algorithms or instructions) that can be executed by the processing unit 2410 in order to carry out different functions (e.g., computer vision-related functionality that would configure the processor of the surgical simulation system to identify relevant points of interest within captured data associated with the user's surgical simulation performance). Furthermore, as discussed above, data such as data sets used to train, validate, and test models used in connection with computer vision and/or machine learning can also be stored here in memory 2415.

It may also be possible, in some embodiments, that some or all of the instructions used to control the processing unit 2410 be instead incorporated into the actual processing unit 2410. In doing so, the instructions incorporated into the processing unit 2410 can then configure it into a special-purpose computing processing unit that is specifically designed to perform the designated function/operations as defined in the incorporated instructions.

All types of data useful for the operation of the surgical simulation system or the like can be stored within memory 2415. In various embodiments, the storage of the data maybe split and stored within computing systems in multiple locations such that some of the data can be stored remotely. However, the data (regardless of where it is physically stored) can still be accessible by the computing system 2400.

The computing system 2400 can also include a cache 2430 that is associated with the processing unit 2410. The cache 2430 may be a separate memory source (though in other embodiments can also be part of the above-described memory 2415) which is temporary in nature. For example, the cache 2430 can be directly connected to, in close proximity with, or integrated to the processing unit 2410. Data that can be stored in the cache 2430 can be accessed much quicker compared to data stored in the above-described memory 2415 (e.g., ROM, RAM, flash).

To enable user interaction with the computing system 2400, input devices 2445 can be used. In general, input devices 2445 can be any number of devices such as a microphone for speech input, A/V sources for audio/visual data, a touch-protected screen for gesture or graphical input, keyboard, mouse, or other types of motion-related input. These type of input devices 2445 can provide the user the capability of providing data for the computing system 2400 to use. In some embodiments, these types of inputs can also allow for the user to adjust/configure various settings associated with the operation of the computing system 2400.

In accordance with various embodiments, the input devices 2445 can correspond to various user manipulable devices (e.g., graspers, probes). These user manipulable devices can have one or more imaging devices that are configured to obtain input from the user, such as captured actions related to a user's performance during a surgical simulation.

In addition, the computing system 2400 can have one or more output devices 2435. These output devices 2435 are configured to provide information to the user related to the surgical simulation. For example, the output devices 2435 may include a display screen that is capable of displaying the feedback from the surgical simulation system related to the user's competency related to a captured surgical simulation performance. The output devices 2435 may also be capable of displaying what is being recorded or captured via the imaging devices or A/V sources in real time for the user. The output devices 2435 may also be separate devices (e.g., lights, speaker, motors, sensors) that are capable of providing real-time feedback to the user during the performance of the surgical simulation (e.g., visual, audio, haptic).

In various embodiments, the computing system 2400 may also be capable of receiving removable storage devices (e.g., USB devices) 2440 that store data and can be accessible by the processing unit 2410. The removable storage devices 2440 may include modules that consist of software that can be used to configure and manage the operation of the processing unit 2410 (and in turn the computing system 2400). The removable storage device 2440 can provide a means by which a user can update or provide additional functionality to the computing system 2400 outside of the existing modules stored within either the memory 2415 or cache 2430.

In different embodiments, the computing system 2400, comprising both of hardware, software, and/or firmware, can take on a variety of different form factors. Exemplary form factors may include personal computers and laptops. However, other form factors that are also possible and contemplated herein include smart phones, rack-mounted devices, and standalone devices.

In some embodiments, parts of the above-described computing system 2400 and/or in general, the surgical simulation network systems and/or portions thereof may be implemented remotely and/or distributive, e.g., in processing and/or assessing surgical simulation performances. For example, some of the computing system 2400 and/or in general, the surgical simulation network system and/or portions thereof may be implemented in the “cloud” thereby allowing for the possibility of having more resources capable of such processing and assessment. This also allows access to additional resources (e.g., processing) which may not be available at the specific computing system 2400 but which can still be used to carry out the functionality being performed by the computing system 2400. Likewise, operations, processes, steps or the like performed by and/or data stored or accessed by the computing system 2400 and/or in general, the surgical simulation network system and/or portions thereof may be distributed among the different computing systems or systems and/or modules within the surgical simulation network system.

Although the present invention has been described in certain specific aspects, many additional modifications and variations would be apparent to those skilled in the art. It is therefore to be understood that the present invention may be practiced otherwise than specifically described without departing from the scope and spirit of the present invention. For example, one of ordinary skill in the art should be able to use the examples to derive a variety of different implementations which retain the main functionalities of the surgical simulation network system described throughout the present disclosure. Although some of the embodiments utilize descriptions which are specific to structural and/or method-related steps, it is to be understood that such subject matter are not necessarily limited to the specifics (e.g., functionality for a feature can be distributed differently over more than one component or be performed in a combination of components different from what was identified explicitly above). Thus, embodiments of the present invention should be considered in all respects as illustrative and not restrictive.

The above description is also provided to enable any person skilled in the art to make and use the devices or systems and perform the methods described herein and sets forth the best modes contemplated by the inventors of carrying out their inventions. Various modifications, however, will remain apparent to those skilled in the art. It is contemplated that these modifications are within the scope of the present disclosure. Different embodiments or aspects of such embodiments may be shown in various figures and described throughout the specification. However, it should be noted that although shown or described separately each embodiment and aspects thereof may be combined with one or more of the other embodiments and aspects thereof unless expressly stated otherwise. It is merely for easing readability of the specification that each combination is not expressly set forth.

1. A surgical simulation network system comprising:
a plurality of surgical trainers configured to capture data associated with a user's performance on one or more surgical simulations;
a database configured to store the data associated with user performance on the one or more surgical simulations; and
a computing system configured to:
process the data associated with user performance on the one or more surgical simulations,
display the data for one or more accessors assigned to review the data,
provides guidelines for the one or more accessors used to assess the data, and
receive feedback from the one or more accessors related to the data.

2. The surgical simulation network system of claim 1, wherein the plurality of surgical trainers further comprise audio and/or video sources that are configured to capture the data associated with the user's performance on the one or more surgical procedures.

3. The surgical simulation network system of claim 2, wherein the audio and/or videos sources comprise sources that are located:
in an internal cavity of each of the plurality of surgical trainers,
in an external space of each of the plurality of surgical trainers, or
both internally and externally for each of the plurality of surgical trainers.

4. The surgical simulation network system of claim 3, wherein the audio and/or video sources located in the internal cavity of each of the plurality of surgical trainers are configured to capture images that track movement of surgical devices and user interaction between the surgical devices and a model enclosed within each of the surgical trainers.

5. The surgical simulation network system of claim 3, wherein the audio and/or video sources located in an external space of each of the plurality of surgical trainers is configured to capture images associated with body posture and hand movements of the user.

6. The surgical simulation network system of claim 3, wherein the captured data associated with a user's performance on one or more surgical simulations comprise a combination of the captured images obtained from the audio and/or video sources located both internal and external to the surgical trainer.

7. The surgical simulation network system of claim 1, wherein each of the plurality of surgical trainers are located at different physical locations.

8. The surgical simulation network system of claim 7, wherein the computing system is located at a different physical location than each of the plurality of surgical trainers.

9. The surgical simulation network system of claim 7, wherein the computing system is cloud-based.

10. The surgical simulation network system of claim 9, wherein one or more of the plurality of surgical trainers are configured to directly stream the captured data associated with a user's performance on one or more surgical simulations to the cloud-based computing system.

11. The surgical simulation network system of claim 9, wherein the cloud-based computing system is further configured to validate the captured data that is directly streamed to ensure that the captured data is satisfactory.

12. The surgical simulation network system of claim 11, wherein the validation of the captured data comprises:
determining that the captured data associated with a user's performance on one or more surgical simulations is of a pre-determined format;
determining that the captured data associated with a user's performance on one or more surgical simulations is of a pre-determined size;
determining that the captured data is complete; and
determining that data packets associated with the captured data are in a correct order.

14. The surgical simulation network system of claim 1, wherein one or more of the plurality of surgical trainers are configured to receive interchangeable models corresponding to different simulated surgical procedures.

15. The surgical simulation network system of claim 1, wherein one or more of the plurality of surgical trainers are configured to receive interchangeable models corresponding to different simulated surgical procedures.

16. The surgical simulation network system of claim 1, wherein the computing system is configured to further tag or link data to the data associated with user performance on the one or more surgical simulations.

17. The surgical simulation network system of claim 16, wherein the tagging or linking is performed manually.

18. The surgical simulation network system of claim 16, wherein the tagging or linking is performed by the computing system automatically via computer vision.

19. The surgical simulation network system of claim 1, wherein the computing system further determines a feedback automatically for the data associated with user performance on the one or more surgical simulations via use of computer vision and machine-learning associated processes.

20. The surgical simulation network system of claim 1, wherein the computing system is configured to provide different guidelines based on an experience the user has when performing the one or more surgical simulations.
